(19) United States
(12) Patent Application Publication
(10) Pub. No.: US 2024/0002503 A1
(43) Pub. Date: Jan. 4, 2024

(54) NOVEL ANTI-LILRB2 ANTIBODIES AND DERIVATIVE PRODUCTS

(71) Applicant: IMMUNE-ONC THERAPEUTICS, INC., Palo Alto, CA (US)

(72) Inventors: Maria Jose COSTA, Palo Alto, CA (US); Ryan STAFFORD, Foster City, CA (US); Krista MCCUTCHEON, Burlingame, CA (US); Jing-Tyan MA, Redwood City, CA (US); Kyu Hee HONG, Palo Alto, CA (US); Hongyu TIAN, Palo Alto, CA (US); An SONG, Palo Alto, CA (US); X. Charlene LIAO, Palo Alto, CA (US)

(21) Appl. No.: 18/249,966

(22) PCT Filed: Oct. 20, 2021
(86) PCT No.: PCT/US2021/055927

§ 371 Date: Apr. 20, 2023

Related U.S. Application Data
(60) Provisional application No. 63/094,354, filed on Oct. 21, 2020.
(60) Provisional application No. 63/110,317, filed on Nov. 5, 2020.

(51) Int. Cl. C07K 16/28; C12N 15/63; G01N 33/574; A61P 35/00; A61K 39/00; C12N 5/078

(52) U.S. Cl. CPC C07K 16/2803; C12N 15/63; G01N 33/574; A61P 35/00; A61K 39/4631; A61K 39/461; C12N 5/0634; C07K 2317/24; C07K 2317/31

(57) ABSTRACT

The present disclosure provides anti-LILRB2 antibodies or antigen-binding fragments thereof, anti-LILRB2 chimeric antigen receptor protein, isolated polynucleotides encoding the same, pharmaceutical compositions comprising the same, and the uses thereof.

### CROSS-REFERENCE TO RELATED APPLICATIONS

This application claims priority to U.S. provisional patent applications No. 63/094,354, filed Oct. 21, 2020, and 63/110,317, filed Nov. 5, 2021, the disclosure of which is incorporated herein by reference.

### SEQUENCE LISTING

The sequence listing that is contained in the file named “066564-8014WO01_ST25”, which is 154 KB (as measured in Microsoft Windows) and was created on Oct. 20, 2021, is filed herewith by electronic submission and is incorporated by reference herein.

### BACKGROUND

### I. Field

The present disclosure relates generally to the fields of medicine, oncology, and immunology. More particular, the disclosure relates to antibodies that bind to LILRB2.

### II. Description of Related Art

Myeloid-derived suppressor cells and tumor-associated macrophages inhibit anti-cancer immune responses systemically and in the tumor microenvironment, respectively, thereby limiting the efficacy of immune checkpoint blockers. On the other hand, the plasticity of myeloid cells may enable therapeutic intervention. Leukocyte Immunoglobulin-Like Receptor subfamily B member 2 (LILRB2), also known as Immunoglobulin-like transcript 4 (ILT4 or ILT-4), Leukocyte Immunoglobulin-like Receptor 2 (LIR2 or LIR-2), and CD85d or CD85D, is a type I membrane protein that is expressed primarily by myeloid cells (monocytes, macrophages, dendritic cells and neutrophils) and has emerged as a key immune checkpoint mediating the tolerogenic activity of myeloid cells associated with cancer. LILRB2 contains cytoplasmic immunoreceptor tyrosine-based inhibition motifs (ITIM) and is involved in negative regulation of immune cell activation. LILRB2 has several ligands that may play a role in cancer (classical and non-classical MHC-I, ANGPTL2/5, SEMA4A, complement split products [CSPs] and CD1c/d), and most of these are known to contribute to immune suppression in the solid tumor microenvironment. Binding of LILRB2 to its ligands results in an inhibitory signal that counteracts stimulation of an immune response. Physiologically, its activity is thought to control inflammatory responses and immune cytotoxicity to help focus the immune response and limit autoreactivity. Thus, LILRB2 is a promising target to modulate immune responses in the treatment of various diseases and conditions, including cancer, chronic viral infections, and autoimmune diseases. There is a significant need for novel anti-LILRB2 antibodies.

### BRIEF SUMMARY OF THE INVENTION

The present disclosure provides anti-LILRB2 antibodies and antigen-binding fragment thereof, amino acid and nucleotide sequences thereof, anti-LILRB2 chimeric antigen receptors, and uses thereof.

In one aspect, the present disclosure provides a monoclonal antibody or an antigen-binding fragment thereof that binds specifically to LILRB2. In certain embodiments, the antibody or antigen-binding fragment, when bound to LILRB2, modulates the activation of LILRB2. In certain embodiments, the antibody or antigen-binding fragment, when bound to LILRB2, suppresses activation of LILRB2. In certain embodiments, the antibody or antigen-binding fragment, when bound to LILRB2, specifically blocks binding of MHC and other ligands (e.g., ANGPTLs, SEMA4A, etc.) to LILRB2.

In some embodiments, the anti-LILRB2 antibody or an antigen-binding fragment thereof comprises a clone-paired heavy chain variable region and light chain variable region as set forth in FIG. 1. In some embodiments, the antibody or antigen-binding fragment thereof comprises (a) the heavy chain variable region has an amino acid sequence of SEQ ID NO: 25 and the light chain variable region has an amino acid sequence of SEQ ID NO: 26; or (b) the heavy chain variable region has an amino acid sequence of SEQ ID NO: 31 and the light chain variable region has an amino acid sequence of SEQ ID NO: 32.

In certain embodiments, the antibody described herein is a recombinant fully human antibody. In certain embodiments, the antibody described herein is of the human IgG1, IgG2, IgG3 or IgG4 type. In certain embodiments, the antigen-binding fragment described herein is a recombinant scFv (single chain fragment variable) antibody, Fab fragment, F(ab′)2 fragment, or Fv fragment.

In certain embodiments, the isolated recombinant fully human antibody described herein is linked to one or more conjugate moieties. In some embodiments, the conjugate moiety comprises an anti-tumor drug, a STING (Stimulator of Interferon Genes) agonist, a cytokine, a clearance-modifying agent, a toxin (e.g., a chemotherapeutic agent), an immune cell stimulator (e.g., a TLR agonist), a detectable label (e.g., a radioactive isotope, a lanthanide, a luminescent label, a fluorescent label, or an enzyme-substrate label), a DNA, an RNA, or purification moiety.

In another aspect, there is provided an isolated nucleic acid that encodes the isolated recombinant fully human antibody or an antigen-binding fragment thereof as provided herein.

In another aspect, there is provided a vector comprising the isolated nucleic acid as provided herein.

In another aspect, there is provided a host cell comprising the vector as provided herein. The host cell may be a mammalian cell. The host cell may be a CHO cell.

In another aspect, there is provided a process of producing an antibody. The method may comprise culturing the host cell as provided herein under conditions suitable for expressing the antibody and recovering the antibody.

In another aspect, there is provided a chimeric antigen receptor (CAR) protein comprising an antigen-binding fragment as provided herein.

In another aspect, there is provided an isolated nucleic acid that encodes a CAR protein as provided herein.

In another aspect, there is provided an engineered cell comprising the isolated nucleic acid as provided herein. In certain embodiments, the cell is a T cell, NK cell, or myeloid cell.

In another aspect, there is provided a method of treating or ameliorating the effect of a cancer or chronic viral infection in a subject, the method comprising administering to the subject a therapeutically effective amount of the antibody or an antigen-binding fragment thereof as defined herein. The method may reduce or eradicate the tumor burden in the subject, may reduce the number of tumor cells, may reduce tumor size, may reduce tumor invasion, may reduce tumor metastasis, may eradicate the tumor in the subject. The cancer may be a solid tumor or hematologic malignancy.

In certain embodiments, the cancer is a solid tumor including adrenal cancer, bile duct carcinoma, bone cancer, brain cancer, breast cancer, cervical cancer, choriocarcinoma, colon cancer, colorectal cancer, esophageal cancer, gastroesophageal junction adenocarcinoma (GEA), eye cancer, gastric cancer, glioblastoma, head and neck cancer, kidney cancer, liver cancer, lung cancer, mesothelioma, melanoma, Merkel cell cancer, nasopharyngeal carcinoma, neuroblastoma, oral cancer, ovarian cancer, pancreatic cancer, penile cancer, pinealoma, prostate cancer, renal cell cancer, retinoblastoma, sarcoma, skin cancer, testicular cancer, thymic carcinoma, thyroid cancer, uterine cancer, and vaginal cancer.

In some embodiments, the cancer is a metastatic, relapsed or drug-resistant cancer.

In some embodiments, said cancer is a hematologic malignancy, including acute lymphocytic leukemia (ALL), acute myeloid leukemia (AML), B-cell leukemia, blastic plasmacytoid dendritic cell neoplasm (BPDCN), chronic lymphoblastic leukemia (CLL), chronic myelomonocytic leukemia (CMML), chronic myelocytic leukemia (CML), pre-B acute lymphocytic leukemia (Pre-B ALL), diffuse large B-cell lymphoma (DLBCL), extranodal NK/T-cell lymphoma, hairy cell leukemia, HHV8-associated primary effusion lymphoma, plasmablastic lymphoma, primary CNS lymphoma, primary mediastinal large B-cell lymphoma, T-cell/histiocyte-rich B-cell lymphoma, heavy chain disease, Hodgkin's lymphoma, non-Hodgkin's lymphoma, Waldenstrom's macroglobulinemia, multiple myeloma (MM), myelodysplastic syndromes (MDS), myeloproliferative neoplasms, and polycythemia vera.

Examples of cancers applicable to methods of treatment herein include but are not limited to, carcinoma, lymphoma, blastoma, sarcoma, and leukemia. More particular nonlimiting examples of such cancers include squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, prostate cancer, vulval cancer, thyroid cancer, hepatic carcinoma. brain cancer, endometrial cancer, testis cancer, cholangiocarcinoma, gallbladder carcinoma, gastric cancer, melanoma, and various types of head and neck cancer (including squamous cell carcinoma of the head and neck).

The antibody or an antigen-binding fragment thereof may be administered intravenously, intra-arterially, intra-tumorally, intra-muscularly or subcutaneously.

In certain embodiments, the method may further comprise administering to the subject one or more drugs selected from the group consisting of a chemotherapeutic agent, a tumor growth inhibitory agent, a cytotoxic agent, an agent used in radiation therapy, an anti-angiogenesis agent, a cancer immunotherapeutic agent, an apoptotic agent, an anti-tubulin agent, a microtubule inhibitor, an anti-HER-2 antibody, an anti-CD20 antibody, an epidermal growth factor receptor (EGFR) antagonist, HER1/EGFR inhibitor, a platelet derived growth factor inhibitor, a COX-2 inhibitor, an interferon, a CTLA4 inhibitor (e.g., anti-CTLA antibody ipilimumab (YERVOY®), or tremelimumab), a PD-1 or PD-L1 inhibitor (e.g., OPDIVO® or nivolumab, KEYTRUDA® or pembrolizumab, TECENTRIQ® or atezolizumab, BAVENCIO® or avelumab, IMFINZI® or durvalumab, LIBTAYO® or cemiplimab-rwlc, TYVYT® or sintilimab, tislelizumab (BGB-A317), penpulimab (AK105), camrelizumab, toripalimab, zimberelimab (GLS-010), retifanlimab, sugemalimab, or CS1003), a dual-targeting antibody against CTLA-4 and PD-1 or PD-L1 (e.g., an anti-PD-1/CTLA-4 bi-specific antibody or AK104), a TIM3 inhibitor (e.g., anti-TIM3 antibodies), a LAG-3 inhibitor (e.g., anti-LAG3 antibodies), a cytokine, an antagonist (e.g., neutralizing antibodies) that bind to one or more of the following targets ErbB2, ErbB3, ErbB4, PDGFR-beta, BlyS, APRIL, BCMA, or VEGF receptor(s), TRAIL/Apo2, an IDH1 inhibitor, an ivosidenib, Tibsovo®, an IDH2 inhibitor, an enasidenib, Idhifa®, a smoothened (SMO) inhibitor, a glasdegib, an arginase inhibitor, an IDO inhibitor, an epacadostat, a BCL-2 inihbitor, a venetoclax, Venclexta®, a platinum complex derivative, oxaliplatin, a kinase inhibitor, a tyrosine kinase inhibitor, a PI3 kinase inhibitor, a BTK inhibitor, an ibrutinib, IMBRUVICA®, an acalabrutinib, CALQUENCE®, a zanubrutinib, an ICOS antibody, a TIGIT antibody, an OX40 antibody, a Toll-like receptor (TLR) agonist, a STING agonist, a TNFR2 antibody, a CD40 antibody, a 4-1BB antibody, a CD47 antibody, a SIRPla antibody or fusions protein, a Siglec antibody, an antibody to another LILR family member, an antagonist of E-selectin, an antibody binding to a tumor antigen, an antibody binding to a T cell surface marker, an antibody binding to a myeloid cell or NK cell surface marker, an alkylating agent, a nitrosourea agent, an antimetabolite, an antitumor antibiotic, an alkaloid derived from a plant, a hormone therapy medicine, a hormone antagonist, an aromatase inhibitor, and a P-glycoprotein inhibitor, an engineered T cell, NK cell or macrophage, a bi-specific antibody.

The isolated fully human recombinant antibody or an antigen binding fragment thereof may comprise an antitumor drug linked thereto. The antitumor drug may be linked to said antibody through a photolabile linker. The antitumor drug may be linked to said antibody through an enzymatically, acid-sensitive or glutathione-sensitive cleavable linker. The antitumor drug may be linked to said antibody through a non-cleavable linker. The antitumor drug may a toxin, a radioisotope, a cytokine, a STING agonist or an enzyme.

In another embodiment, there is provided a method of detecting a cancer cell or cancer stem cell in a sample or subject comprising (a) contacting a subject or a sample from the subject with the antibody or an antigen-binding fragment thereof as defined herein; and (b) detecting binding of said antibody to a cancer cell or cancer stem cell in said subject or sample. The sample may be a body fluid or biopsy, or blood, bone marrow, sputum, tears, saliva, mucous, serum, ascites, urine or feces. Detection may comprise immunohistochemistry, flow cytometry, immunoassays (including ELISA, RIA etc.) or Western blot. The method may further comprise performing steps (a) and (b) a second time and determining a change in detection levels as compared to the first time. The isolated recombinant antibody or an antigen binding fragment thereof may further comprise a label, such as a peptide tag, an enzyme, a magnetic particle, a chromophore, a fluorescent molecule, a chemo-luminescent molecule, or a dye. The isolated recombinant antibody or an antigen binding fragment thereof may be conjugated to a liposome or nanoparticle.

In still an additional aspect, there is provided a method of treating or ameliorating the effect of chronic viral infection in a subject, the method comprising administering to the subject a therapeutically effective amount of the antibody or an antigen-binding fragment thereof as defined herein. The antibody or an antigen-binding fragment thereof may be administered intravenously, intra-arterially, or subcutaneously. In some embodiments, the chronic viral infection is caused by a virus selected from Herpes Simplex I (HSV-I), Herpes Simplex II (HSV-II), Herpes Virus 3, Herpes Virus 4, Herpes Virus 5, Herpes Virus 6, Parvo Virus B19, Coxsackie A & B, Hepatitis A, Hepatitis B, Hepatitis C, Cytomegalovirus (CMV), and Human Immunodeficiency Virus (HIV).

### BRIEF DESCFRIPTION OF FIGURES

The following drawings form part of the present specification and are included to further demonstrate certain aspects of the present invention. The invention may be better understood by reference to one or more of these drawings in combination with the detailed description of specific embodiments presented herein.

FIG. 1 shows the heavy chain variable region (VH) and light chain variable region (VL) amino acid sequences of certain anti-LILRB2 antibodies derived from antibody B2-19 which has been disclosed in PCT Patent Application No. PCT/US2021/015362.

FIG. 2 shows that the B2-19 antibody variants B2-19-12 and B2-19-16 have the same binding affinity to LILRB2 as the parent B2-19 antibody. The binding affinities of B2-19 and select B2-19-derived variants to recombinant LILRB2 extracellular domain (ECD) protein (with 6× His tag at C-terminus) were measured by Bio-Layer Interferometry (BLI). The measured binding affinities were all very similar, within experimental error, with a K_{D} of approximately 2.0 nM.

FIG. 3 shows that the B2-19 antibody and its variants have comparable binding potency to LILRB2 stably expressed on HEK293 cells. Binding potency (EC₅₀) of LILRB2 antibodies to HEK293 cells stably expressing LILRB2 was determined by flow cytometry.

FIG. 4 shows that the B2-19 antibody and its variants have comparable binding potency (EC₅₀) to endogenous LILRB2 expressed on primary CD14⁺CD16⁻ monocytes isolated from healthy donors' peripheral blood mononuclear cells (PBMC).

FIG. 5 shows that the B2-19 antibody and its variants bind specifically to LILRB2. The binding specificity of the antibodies to LILRB2 was analyzed by ELISA.

FIG. 6 shows that the B2-19 antibody and its variants bind specifically to myeloid cells in human whole blood. The reactivity of LILRB2 antibodies to leukocytes from whole blood harvested from healthy donors was characterized by flow cytometry. Data shown is corrected geometric mean fluorescence intensity (MFI) of sample, i.e., geometric MFT of anti-LILRB2 stained samples subtracted by geometric MFI of samples in which LILRB2 antibodies were omitted (fluorescence minus one [FMO] control). Representative data from one donor is shown (N=3 donors).

FIG. 7 shows that the B2-19 antibody and its variants have comparable ability to block LILRB2 from binding to HLA-G. The anti-LILRB2 antibodies competitively inhibited HLA-G binding to HEK293 cells stably expressing LILRB2, with potency (IC5o) determined by flow cytometry.

FIGS. 8A and 8B show that the B2-19 and B2-19-16 antibodies have comparable pro-inflammatory effect on PBMC samples isolated from healthy donors and stimulated with an anti-CD3 agonistic monoclonal antibody at sub-optimal concentration. Each line represents the paired results (human IgG4 isotype control antibody vs anti-LILRB2 blocking antibody) from an individual donor and data are pooled from 6 independent experiments. The fraction of donors showing that anti-LILRB2 blocking antibodies produce a detectable change in cytokine production/secretion levels consistent with an enhanced pro-inflammatory effect is shown in between parentheses, *p<0.05, **p<0.001, ***p=0.0001, ****p<0.0001 (paired t test). FIG. 8A shows the cytokine levels of PBMC stimulated with ng/mL anti-CD3 antibody and 15 μg/mL isotype control or B2-19-16. FIG. 8B shows the cytokine levels of PBMC stimulated with 10 ng/mL anti-CD3 antibody and 4 μg/mL or 15 μg/mL (depending on the experiment) isotype control or B2-19.

FIG. 9 shows that B2-19-derived variants demonstrate lower polyspecificity than the parent B2-19 antibody, as evidenced by improved baculolvirus particle (BVP) scores. The graph shows the OD4sonm from the ELISA of antibodies bound to plates coated with 0.5% BVP (v/v stock solution; titer=5.71×10¹² pfu/mL) and using 1/20,000 dilution of anti-human IgG secondary antibody. Literature has shown antibodies with BVP scores 5-fold over background are prone to poor pharmacokinetics in humans and non-human primates (Hotzel et al 2012, mAbs, 753-760). Therefore, B2-19-12 and B2-19-16 are more suitable for therapeutic development than the parent B2-19.

FIG. 10 shows that B2-19 variants do not appreciably lose binding activity after being subjected to thermal stress. Data are dose-response curves and calculated potency (EC₅₀) of binding to LILRB2 as measured by ELISA.

FIG. 11 shows that B2-19 variants do not lose binding activity upon freeze-thaw (F/T). Data are dose-response curves and derived EC₅₀ of antibody binding to LILRB2, as measured by ELISA.

FIG. 12 shows the pharmacokinetics (PK) of B2-19-12 and B2-19-16 in human FcRn transgenic mice. Both B2-19-12 and B2-19-16 exhibit pharmacokinetic parameters within typical ranges for a human IgG in human FcRn transgenic mice.

FIGS. 13A and 13B are flow cytometry data showing that B2-19-16 binds to all myeloid cells infiltrating solid tumor microenvironment, as well as peripheral blood myeloid cells from solid tumor patients. CD11b is used as pan-myeloid cell marker and CD45 is used as pan-tumor-infiltrating leukocytes marker. FIG. 13A shows flow cytometry data from tumor tissue samples of 3 different solid tumor patients. FIG. 13B shows flow cytometry data from peripheral blood of solid tumor patient #3. Black-filled histogram: sample incubated with B2-19-16; white-filled histogram: sample incubated with IgG4 isotype control.

FIG. 14 shows that B2-19-16 antibody further potentiates the effect of lipopolysaccharide (LPS) on the maturation/activation of monocyte-derived dendritic cells, as shown by decreased expression levels of the tolerogenic marker CD209. Each line represents result from a different healthy donor sample in which CD209 levels on cell surface were analyzed by flow cytometry. The fraction of donor samples showing a change in CD209 expression levels consistent with an enhanced pro-inflammatory effect is shown in between parentheses, ****p<0.0001 (paired t test).

FIG. 15 shows that B2-19-16 antibody enhances TNF-α production/secretion when monocyte-derived dendritic cells are matured/activated by LPS stimulation. Each line represents result from a different healthy donor. The fraction of donor samples showing a change in TNF-α concentration levels consistent with an enhanced pro-inflammatory effect is shown in between parentheses, *p<0.05, (paired t test).

FIGS. 16A and 16B show that B2-19-16 antibody promotes the differentiation of primary monocytes into activated (CD86⁺) dendritic cells (DCs). The effects of anti-LILRB2 antibodies on the in vitro differentiation of monocytes into DCs were analyzed by flow cytometry. FIG. 16A shows the flow cytometry data from 2 healthy donor samples and the values indicated in each histogram represent the percent of CD86⁺ DCs obtained at the end of a 6-day culture in each experimental condition. FIG. 16B shows the combined results from all 7 analyzed donors, **p=0.002 (paired t test).

FIG. 17 is flow cytometry data showing that B2-19-16 enhances the expression levels of maturation (CD83) and activation markers (CD86, HLA-DR) in immature monocyte-derived DCs while decreasing expression levels of the tolerogenic marker CD209. The expression levels of LILRB4, another immune inhibitory receptor, remains unchanged. Each line represents result from a different healthy donor sample, *p<0.05, **p<0.008, n. s.=non-significant (paired t test).

FIG. 18 shows that B2-19-16 enhances IFN-γ production/secretion in allogeneic CD4⁺ T cell-macrophage co-cultures stimulated by an anti-PD-1 blocking antibody. Each line represents results from one allogeneic CD4⁺ T cell-macrophage co-culture, p=0.0032 (repeated measures, one-way ANOVA).

FIG. 19 shows that B2-19-16 enhances the production/secretion of multiple pro-inflammatory cytokines in LPS-stimulated PBMC samples derived from healthy donors, while decreasing the production/secretion of the anti-inflammatory cytokine IL-10. Each line represents the paired results from an individual donor sample and data are pooled from 4 independent experiments. Fraction of donor samples showing a change in cytokine concentration levels consistent with an enhanced pro-inflammatory effect is shown in between parentheses, **p<0.001 (paired t test).

FIG. 20 shows that B2-19-16 antibody enhances TNF-α production/secretion levels by LPS-stimulated PBMC in a dose-dependent manner. Results are representative of 5 PBMC donor samples.

FIG. 21 shows that B2-19-16 antibody enhances TNF-α production/secretion by monocyte-derived macrophages stimulated with STING agonist 2′3′-cGAMP in all tested donor samples.

FIGS. 22A and 22B show that B2-19-16 antibody reverts the tolerogenic phenotype of PBMC-derived myeloid cells (CD33 +) caused by “tumor-conditioning.” Each line corresponds to results from one myeloid cell healthy donor. FIG. 22A shows the result of co-culture with SK-MEL-5 melanoma-derived cell line on the immunophenotyping of myeloid cells. FIG. 22B shows the result of co-culture with A549 lung adenocarcinoma-derived cell line on the immunophenotyping of myeloid cells.

FIG. 23 shows that B2-19-16 does not induce internalization of LILRB2 in monocyte-derived macrophages from two healthy donors. An anti-CD71 (transferrin receptor) antibody was used as a positive control for internalization of receptor:antibody complexes. Internalization was monitored for up to 12 hours using an Incucyte Live-Cell Analysis system.

FIG. 24 shows that B2-19-16 does not trigger Fc-mediated depletion of LILRB2⁺ cells (monocytes from PBMC) in vitro. To evaluate the ability of B2-19-16 to trigger Fc-mediated monocyte depletion, PBMC were incubated with up to 40 μg/mL B2-19-16 (or isotype control) for 20 hours, but no decrease in monocyte viability was observed. In contrast, rituximab, a known B cell depleting IgG1 antibody, potently decreased B cell viability in a parallel incubation with PBMC sample from the same donors.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### I. Definitions

It is to be understood that both the foregoing general description and the following detailed description are exemplary and explanatory only and are not restrictive of the invention as claimed. In this application, the use of the singular includes the plural unless specifically stated otherwise. In this disclosure, the term “or” is used to mean “and/or” unless explicitly indicated to refer to alternatives only or the alternatives are mutually exclusive. As used herein “another” may mean at least a second or more. Furthermore, the use of the term “including”, as well as other forms, such as “includes” and “included”, is not limiting. Also, terms such as “element” or “component” encompass both element or component comprising one unit and elements or components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise.

The term “about” as used herein when referring to a measurable value such as an amount, a temporal duration, and the like, is meant to encompass variations of up to ±10% from the specified value. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the disclosed subject matter. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The term “antibody” as used herein includes any immunoglobulin, monoclonal antibody, polyclonal antibody, multivalent antibody, bivalent antibody, monovalent antibody, multi-specific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable domain (V H) and a constant region including a first, second, and third constant domain (C_{H1}, C_{H2}, C_{H3}, respectively); mammalian light chains are classified as λ or κ, while each light chain consists of a variable domain (V L) and a constant domain (CO. A typical IgG antibody has a “Y” shape, with the stem of the Y typically consisting of the second and third constant domains of two heavy chains bound together via disulfide bonding. Each arm of the Y includes the variable domain and first constant domain of a single heavy chain bound to the variable and constant domains of a single light chain. The variable domains of the light and heavy chains are responsible for antigen binding. The variable domains in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. (1985) 186(3):651-63; Chothia, C. and Lesk, A. M., J. Mol. Biol. (1987) 196:901; Chothia, C. et al., Nature (1989) 342(6252):877-83; Marie-Paule Lefranc et al., Developmental and Comparative Immunology (2003) 27: 55-77; Marie-Paule Lefranc et al., Immunome Research (2005) 1(3); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant domains of the heavy and light chains are not involved in antigen-binding but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gammal heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

The term “antigen” refers to a substance capable of inducing adaptive immune responses. Specifically, an antigen is a substance specifically bound by antibodies or T lymphocyte antigen receptors. Antigens are usually proteins and polysaccharides, less frequently also lipids. Suitable antigens include without limitation parts of bacteria (coats, capsules, cell walls, flagella, fimbrai, and toxins), viruses, and other microorganisms. Antigens also include tumor antigens, e.g., antigens generated by mutations in tumors. As used herein, antigens also include immunogens and haptens.

The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds-diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

A “Fab fragment” comprises one light chain and the C_{H}1 and variable domains of one heavy chain. The heavy chain of a Fab molecule cannot form a disulfide bond with another heavy chain molecule.

A “Fab′ fragment” comprises one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

“Fv” with regard to an antibody refers to the smallest fragment of the antibody to bear the complete antigen-binding site. An Fv fragment consists of the variable domain of a single light chain bound to the variable domain of a single heavy chain.

“Single-chain Fv antibody” or “scFv” refers to an engineered antibody consisting of a light chain variable domain and a heavy chain variable domain connected to one another directly or via a peptide linker sequence (Huston J S et al., Proc Natl Acad Sci USA (1988) 85:5879).

An “Fc” region comprises two heavy chain fragments comprising the C_{H}2 and C_{H}3 domains of an antibody. The two heavy chain fragments are held together by two or more disulfide bonds and by hydrophobic interactions of the C_{H}3 domains. The Fc region of the antibody is responsible for various effector functions such as antibody-dependent cell-mediated cytotoxicity (ADCC), and complement dependent cytotoxicity (CDC), but does not function in antigen binding.

“Single-chain Fv-Fc antibody” or “scFv-Fc” refers to an engineered antibody consisting of a scFv connected to the Fc region of an antibody.

A “dsFv” refers to a disulfide-stabilized Fv fragment that the linkage between the variable domain of a single light chain and the variable domain of a single heavy chain is a disulfide bond. In some embodiments, a “(dsFv)₂” or “(dsFv-dsFv′)” comprises three peptide chains: two V_{H} domains linked by a peptide linker (e.g., a long flexible linker) and bound to two V_{L} domains, respectively, via disulfide bridges. In some embodiments, dsFv-dsFv′ is bispecific in which each disulfide paired heavy and light chain has a different antigen specificity.

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature (1993) 363:446-8; Nguyen V K. et al., Immunogenetics (2002) 54:39-47; Nguyen V K. et al., Immunology (2003) 109:93-101). The variable domain of a heavy chain antibody (VHH domain) represents the smallest known antigen-binding unit generated by adaptive immune responses (Koch-Nolte F. et al., FASEB J. (2007) 21:3490-8).

A “nanobody” refers to an antibody fragment that consists of a VHH domain from a heavy chain antibody and two constant domains, CH2 and CH3.

“Diabodies” or “dAbs” include small antibody fragments with two antigen-binding sites, wherein the fragments comprise a V_{H} domain connected to a V_{L} domain in the same polypeptide chain (V_{H}-V_{L} or V_{L}-V_{H}) (see, e.g., Holliger P. et al., Proc Natl Acad Sci U S A. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds-diabody” is a diabody target two different antigens (or epitopes).

In certain embodiments, an “scFv dimer” is divalent (or bivalent) single-chain variable fragments (di-scFvs, bi-scFvs) that can be engineered by linking two scFvs. A bivalent diabody or bivalent scFv (BsFv, di-scFvs, bi-scFvs) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with Vₗ₁-V_{H2} (also linked by a peptide linker) such that V_{H1} and V_{L1} coordinate and V_{H2} and V_{L2} coordinate and each coordinated pair has a different antigen specificity.

A “domain antibody” refers to an antibody fragment containing only the variable domain of a heavy chain or the variable domain of a light chain. In certain instances, two or more V_{H} domains are covalently joined with a peptide linker to create a bivalent or multivalent domain antibody. The two V_{H} domains of a bivalent domain antibody may target the same or different antigens.

A “bispecific” antibody refers to an artificial antibody which has fragments derived from two different monoclonal antibodies and is capable of binding to two different epitopes. The two epitopes may present on the same antigen, or they may present on two different antigens.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (K D). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope. For example, the LILRB2 specific antibodies of the present invention are specific to LILRB2. In some embodiments, the antibody that binds to LILRB2 has a dissociation constant (K_{D}) of ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³M). The dissociation constant K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS, BLI method and flow cytometry method. In certain embodiments, the K D value can be appropriately determined by using flow cytometry.

“Cancer” as used herein refers to any medical condition characterized by malignant cell growth or neoplasm, abnormal proliferation, infiltration or metastasis, and includes both solid tumors and non-solid cancers (hematologic malignancies) such as leukemia. As used herein “solid tumor” refers to a solid mass of neoplastic and/or malignant cells. Examples of cancer or tumors include hematological malignancies, oral carcinomas (for example of the lip, tongue or pharynx), digestive organs (for example esophagus, stomach, small intestine, colon, large intestine, or rectum), peritoneum, liver and biliary passages, pancreas, respiratory system such as larynx or lung (small cell and non-small cell), bone, connective tissue, skin (e.g., melanoma), breast, reproductive organs (fallopian tube, uterus, cervix, testicles, ovary, or prostate), urinary tract (e.g., bladder or kidney), brain and endocrine glands such as the thyroid. In certain embodiments, the cancer is selected from ovarian cancer, breast cancer, head and neck cancer, renal cancer, bladder cancer, hepatocellular cancer, and colorectal cancer. In certain embodiments, the cancer is selected from a lymphoma, Hodgkin's lymphoma, non-Hodgkin's lymphoma and B-cell lymphoma.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse or rabbit. In some embodiments, the non-human animal is a mammal, for example, a mouse, a rat, a rabbit, a goat, a sheep, a guinea pig, or a hamster.

The term “specific binding” or “specifically binds” as used herein refers to a non-random binding reaction between two molecules, such as for example between an antibody and an antigen. In certain embodiments, the antibodies or antigen-binding fragments provided herein specifically bind to LILRB2 with a binding affinity (K_{D}) of ≤10⁻⁶M (e.g., ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤4×10^{31 9}M, ≤3×10⁻⁹M, ≤2×10⁻⁹ M, or ≤10⁻⁹ M). K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry method. In certain embodiments, the K_{D} value can be appropriately determined by using flow cytometry.

The ability to “block binding” or to “compete for the same epitope” as used herein refers to the ability of an antibody or antigen-binding fragment to inhibit the binding interaction between two molecules (e.g. LILRB2 and an anti-LILRB2 antibody) to any detectable degree. In certain embodiments, an antibody or antigen-binding fragment that blocks binding between two molecules inhibits the binding interaction between the two molecules by at least 85%, or at least 90%. In certain embodiments, this inhibition may be greater than 85%, or greater than 90%.

Those skilled in the art will recognize that it is possible to determine, without undue experimentation, if a given antibody binds to the same epitope as the antibody of present disclosure by ascertaining whether the former prevents the latter from binding to a LILRB2 antigen polypeptide. If the given antibody competes with the antibody of present disclosure, as shown by a decrease in binding by the antibody of present disclosure to the LILRB2 antigen polypeptide, then the two antibodies bind to the same, or a closely related, epitope. Or if the binding of a given antibody to the LILRB2 antigen polypeptide was inhibited by the antibody of present disclosure, then the two antibodies bind to the same, or a closely related, epitope.

The term “chimeric antigen receptor” or “CAR”, as used herein, refer to engineered receptors that are capable of grafting a desired specificity to an antigen into immune effector cells, such as T cells, NK cells and macrophages. Typically, a CAR protein comprises an extracellular domain that introduces the desired specificity, a transmembrane domain and an intracellular domain that transmits a signal to the immune effector cells when the immune effector cells bind to the antigen. In certain embodiments, the extracellular domain comprises a leader peptide, an antigen recognition region and a spacer region. In certain embodiments, the antigen recognition region is derived from an antibody that specifically binds to the antigen. In certain embodiments, the antigen recognition region is a single-chain variable fragment (scFv) derived from the antibody. In certain embodiments, the single-chain variable fragment (scFv) is derived from a humanized antibody. In certain embodiment, the single-chain variable fragment comprises a heavy chain variable region fused to a light chain variable region through a flexible linker.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

“Effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and phagocytosis.

The term “epitope” as used herein refers to the specific group of atoms or amino acids on an antigen to which an antibody binds. Two antibodies may bind the same or a closely related epitope within an antigen if they exhibit competitive binding for the antigen. For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “homologue” and “homologous” as used herein are interchangeable and refer to nucleic acid sequences (or its complementary strand) or amino acid sequences that have sequence identity of at least 80% (e.g., at least 85%, 88%, 90%, 91%, 92%, 93%, 94%, 95%, 96%, 97%, 98%, 99%) to other sequences when optimally aligned.

The phrase “host cell” as used herein refers to a cell into which an exogenous polynucleotide and/or a vector has been introduced.

The term “humanized” as used herein means that the antibody or antigen-binding fragment comprises CDRs derived from non-human animals, FR regions derived from human, and when applicable, the constant regions derived from human.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or antigen-binding fragment thereof” refers to the antibody or antigen-binding fragments having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

A “leader peptide” refers to a peptide having a length of about 5-30 amino acids that is present at the N-terminus of newly synthesized proteins that form part of the secretory pathway. Proteins of the secretory pathway include, but are not limited to proteins that reside either inside certain organelles (the endoplasmic reticulum, Golgi or endosomes), are secreted from the cell, or are inserted into a cellular membrane. In some embodiments, the leader peptide forms part of the transmembrane domain of a protein.

Leukocyte immunoglobulin-like receptor subfamily B member 2 (LILRB2) is a protein that in humans is encoded by the LILRB2 gene. This gene is a member of the leukocyte immunoglobulin-like receptor (LIR) family, which is found in a gene cluster at chromosomal region 19q13.4. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on immune cells where it binds to MEW class I molecules and other ligands on antigen-presenting cells and transduces a negative signal that inhibits stimulation of an immune response. The receptor can also play a role in antigen capture and presentation. It is thought to control inflammatory responses and cytotoxicity to help focus the immune response and limit autoreactivity. Multiple transcript variants encoding different isoforms have been found for this gene. LILRB2 has been shown to interact with PTPN6.

The term “anti-LILRB2 antibody” refers to an antibody that is capable of specifically binding to LILRB2.

A “LILRB2-related” disease or condition as used herein refers to any disease or condition caused by, exacerbated by, or otherwise linked to increased or decreased expression or activities of LILRB2. In some embodiments, the LILRB2 related condition is immune-related disorder, such as, for example, cancer, autoimmune disease, inflammatory disease or infectious disease.

The term “link” as used herein refers to the association via intramolecular interaction, e.g., covalent bonds, metallic bonds, and/or ionic bonding, or inter-molecular interaction, e.g., hydrogen bond or noncovalent bonds.

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol. (1990) 215:403-410; Stephen F. et al., Nucleic Acids Res. (1997) 25:3389-3402), ClustalW2 (available on the website of European Bioinformatics Institute, see also, Higgins D. G. et al., Methods in Enzymology (1996) 266:383-402; Larkin M. A. et al., Bioinformatics (2007) 23:2947-8), and ALIGN or Megalign (DNASTAR) software. Those skilled in the art may use the default parameters provided by the tool or may customize the parameters as appropriate for the alignment, such as for example, by selecting a suitable algorithm.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The term “polypeptide” or “protein” means a string of at least two amino acids linked to one another by peptide bonds. Polypeptides and proteins may include moieties in addition to amino acids (e.g., may be glycosylated) and/or may be otherwise processed or modified. Those of ordinary skill in the art will appreciate that a “polypeptide” or “protein” can be a complete polypeptide chain as produced by a cell (with or without a signal sequence), or can be a functional portion thereof. Those of ordinary skill will further appreciate that a polypeptide or protein can sometimes include more than one polypeptide chain, for example linked by one or more disulfide bonds or associated by other means. The term also includes amino acid polymers in which one or more amino acids are chemical analogs of a corresponding naturally-occurring amino acid and polymers.

The pharmaceutically acceptable carriers useful in this invention are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms) , conventional non-toxic solid carriers can include, for example, pharmaceutical grades of mannitol, lactose, starch or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective dosage” as used herein refers to the dosage or concentration of a drug effective to treat a disease or condition. For example, with regard to the use of the monoclonal antibodies or antigen-binding fragments thereof disclosed herein to treat cancer, a therapeutically effective amount is the dosage or concentration of the monoclonal antibody or antigen-binding fragment thereof capable of reducing the tumor volume, eradicating all or part of a tumor, inhibiting or slowing tumor growth or cancer cell infiltration into other organs, inhibiting growth or proliferation of cells mediating a cancerous condition, inhibiting or slowing tumor cell metastasis, ameliorating any symptom or marker associated with a tumor or cancerous condition, preventing or delaying the development of a tumor or cancerous condition, or some combination thereof.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or antigen-binding fragment thereof, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

### II. Anti-LILRB2 Antibody and Antigen-Binding Fragment

The present disclosure in one aspect provides anti-LILRB2 antibodies and antigen-binding fragment thereof. In some embodiments, the anti-LILRB2 antibodies and antigen-binding fragment thereof are derived or modified from certain anti-LILRB2 antibodies disclosed in PCT Patent Application No. PCT/US2021/015362 (the disclosure of which is incorporated herein by reference). The anti-LILRB2 antibodies disclosed in PCT Patent Application No. PCT/US2021/015362, in some embodiments, were prepared using phage display method. The phage display method involves, in short, a large library of phage displayed human scFv panned against the target protein, i.e., LILRB2. The human scFv selected to specifically bind to the target protein can be sequenced and then subcloned into a human IgG expression vector to produce the desired fully human antibody.

In some embodiments, the anti-LILRB2 antibodies and antigen-binding fragment thereof disclosed herein, as compared to the anti-LILRB2 antibodies disclosed in PCT Patent Application No. PCT/US2021/015362, retain the ability to bind to LILRB2 with high affinity and specificity, as well as the same potent ligand blocking activity, but have improved properties in terms of developing therapeutic or diagnostic products. In some embodiments, such antibodies and antigen-binding fragment thereof have distinct physicochemical properties as compared to the anti-LILRB2 antibodies disclosed in PCT Patent Application No. PCT/US2021/015362 resulting in improved manufacturability, stability and pharmacokinetic properties. In certain embodiments, such antibodies and antigen-binding fragment thereof have lower immunogenicity in human.

### Specific Anti-LILRB2 Antibodies

In certain embodiments, the anti-LILRB2 antibody disclosed herein is derived from the antibody B2-19 having heavy chain variable region sequence of SEQ ID NO: 1 and light chain variable region sequence of SEQ ID NO: 2. In particular embodiments, the anti-LILRB2 antibodies disclosed herein have enhanced manufacturability, stability and/or and pharmacokinetic profile as compared to B2-19, yet substantially retain the ability to bind to LILRB2 with the same level of specificity and affinity. Additionally, the anti-LILRB2 antibodies disclosed herein retain the same ligand blocking activity.

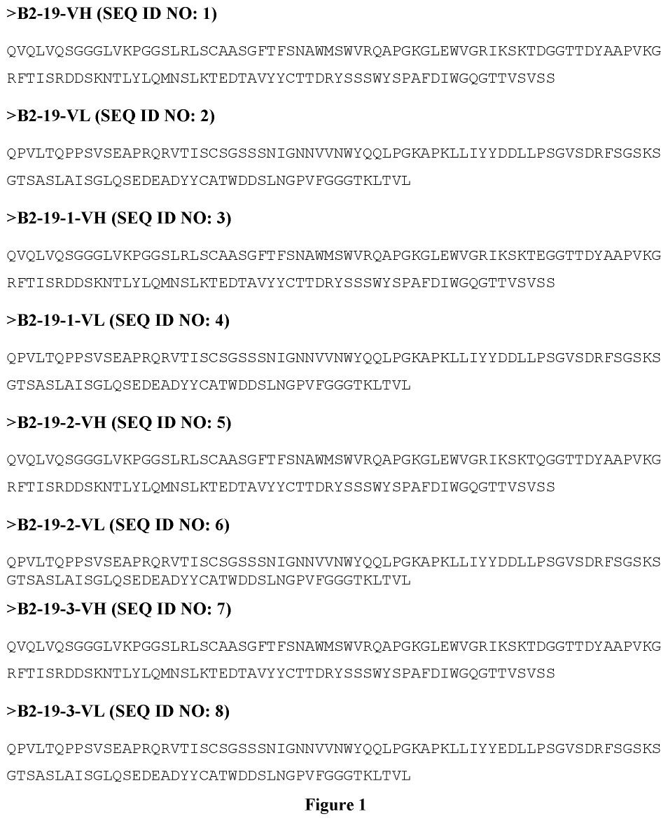

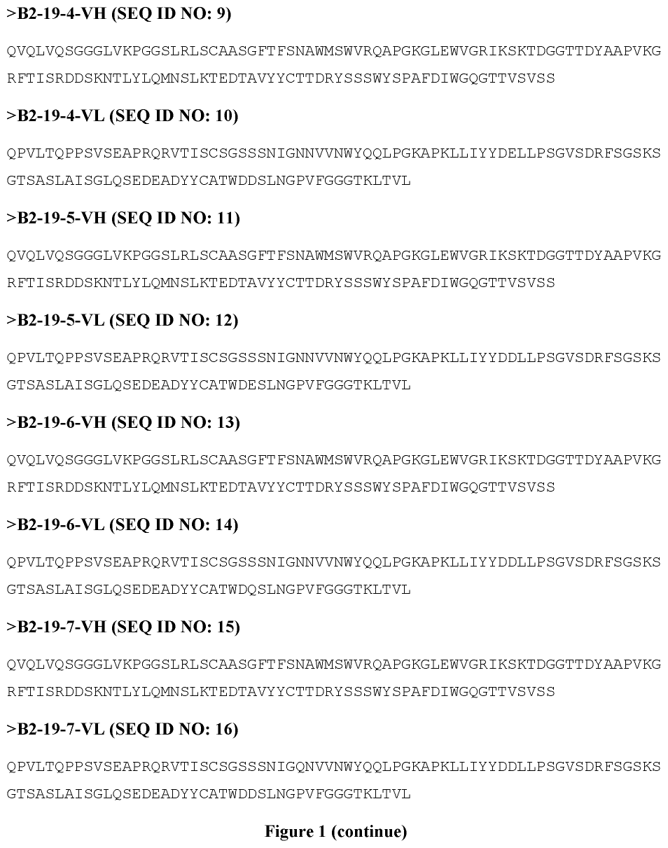

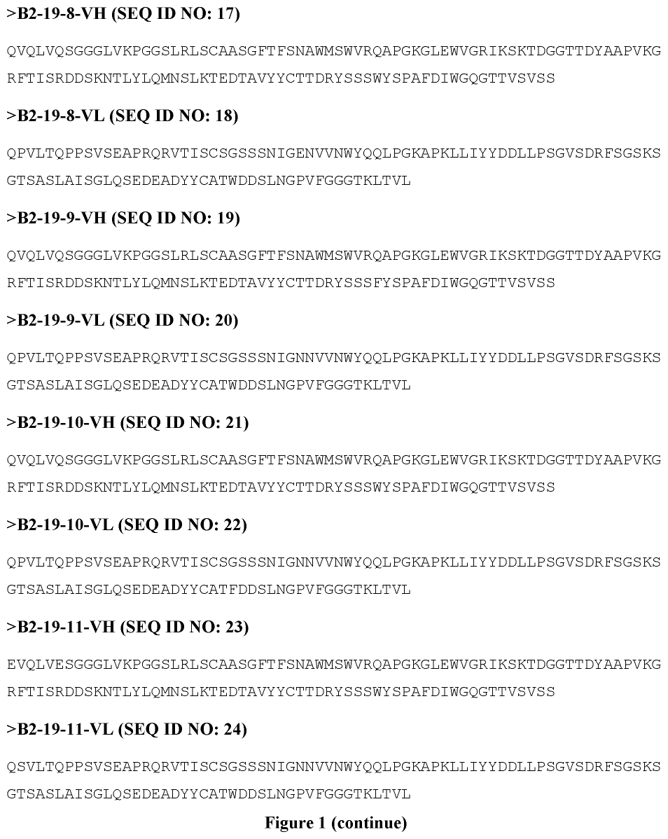

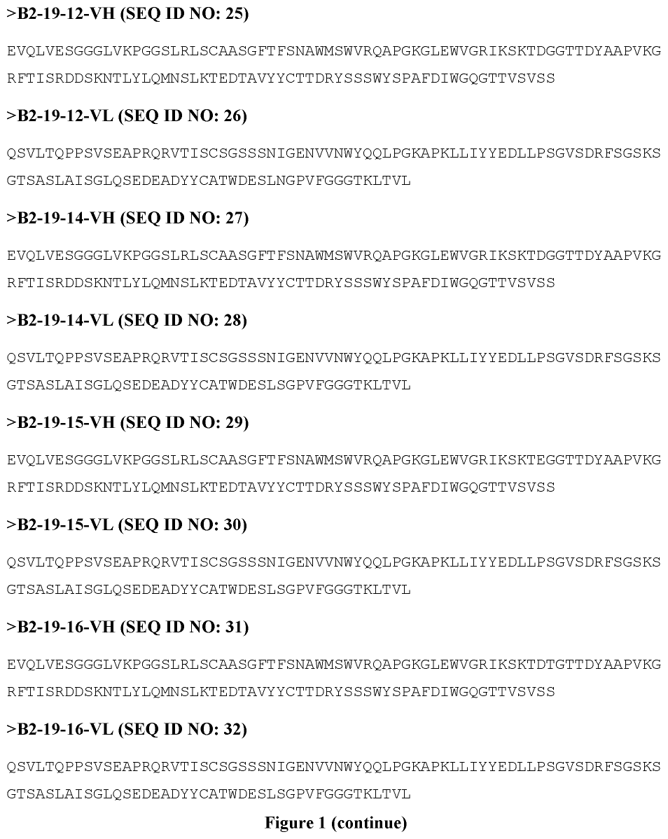

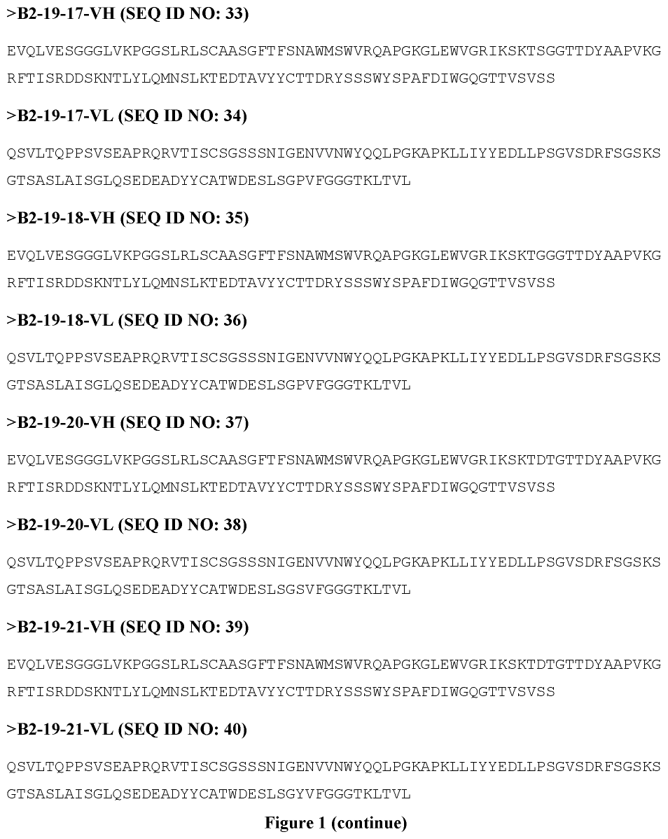

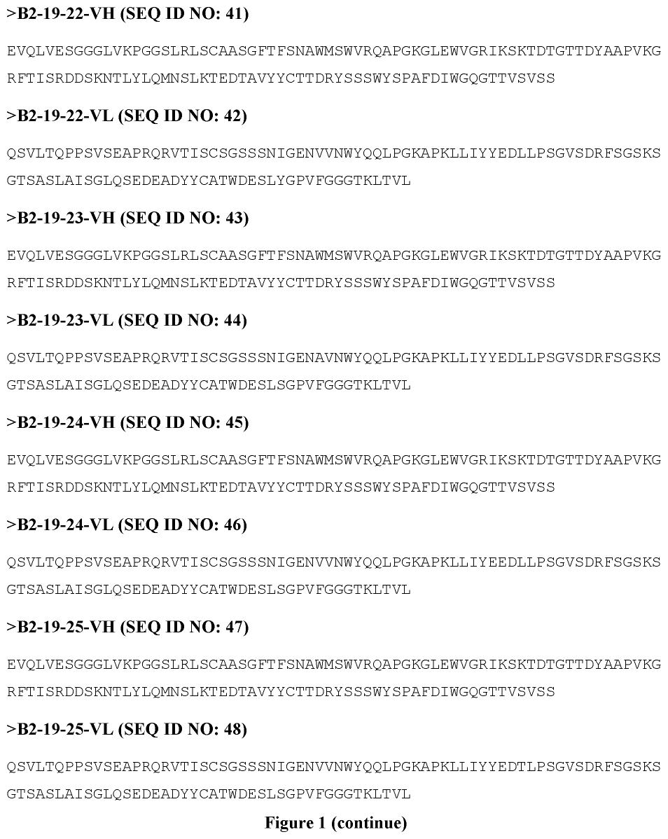

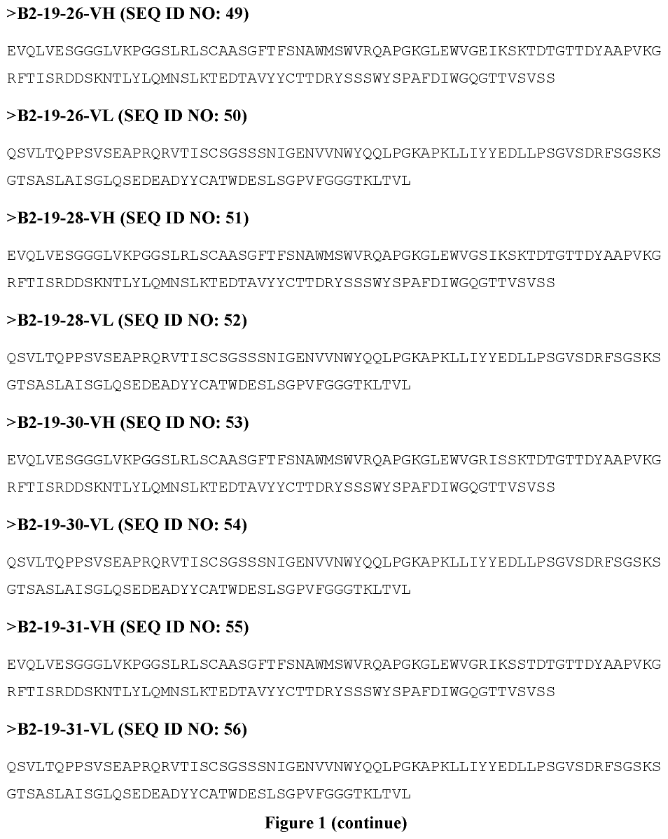

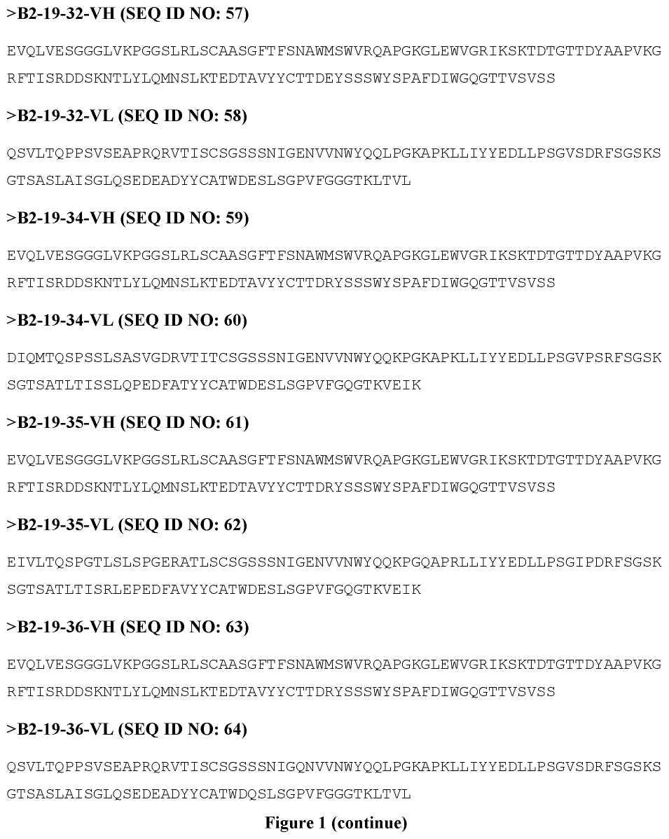

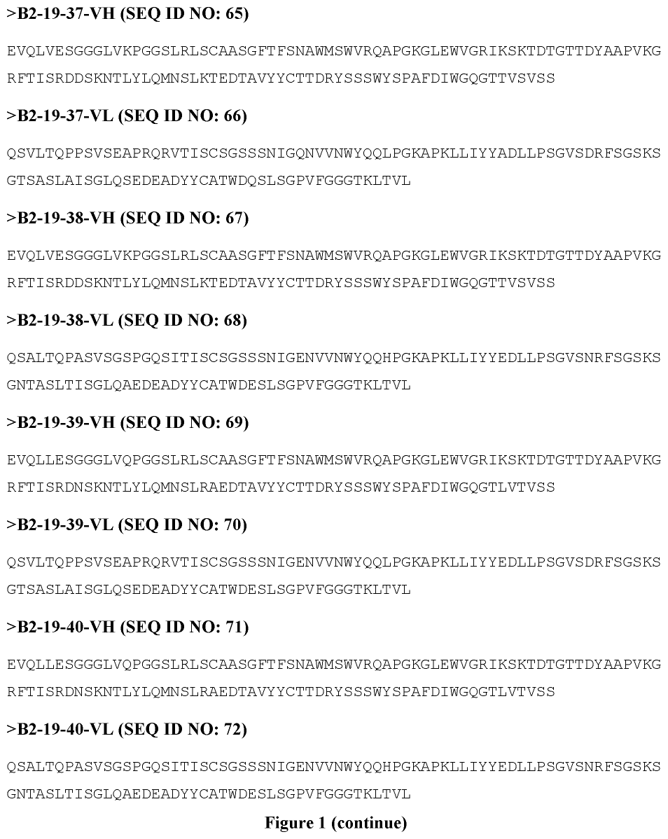

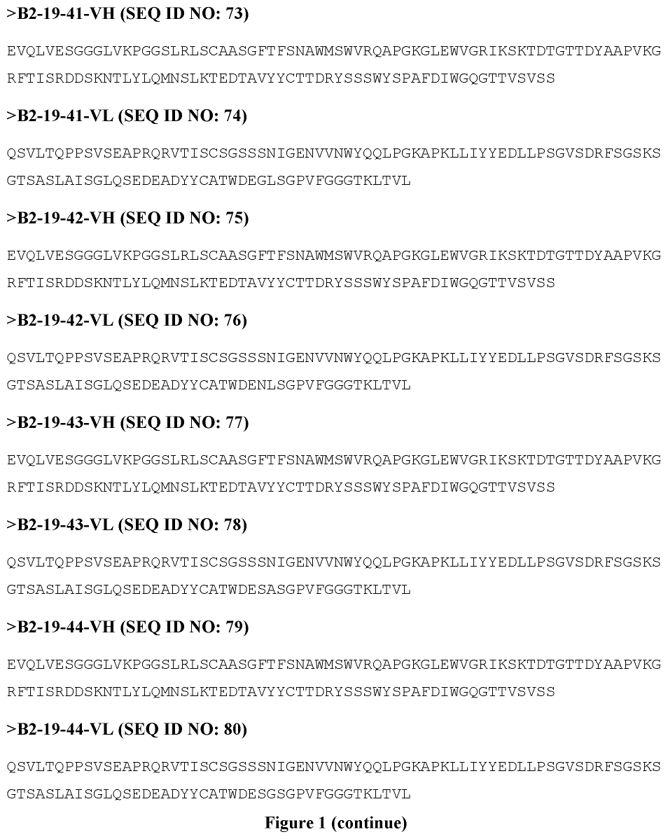

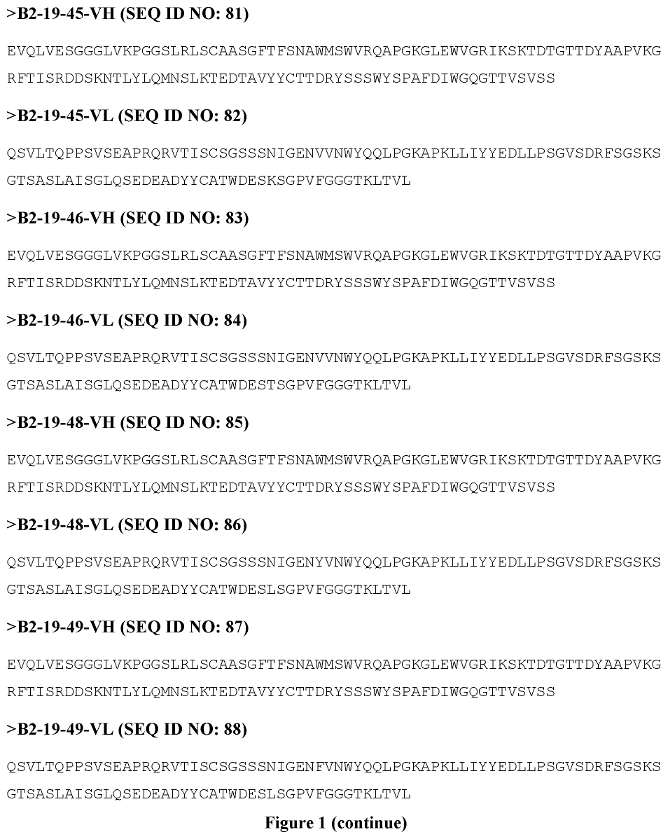

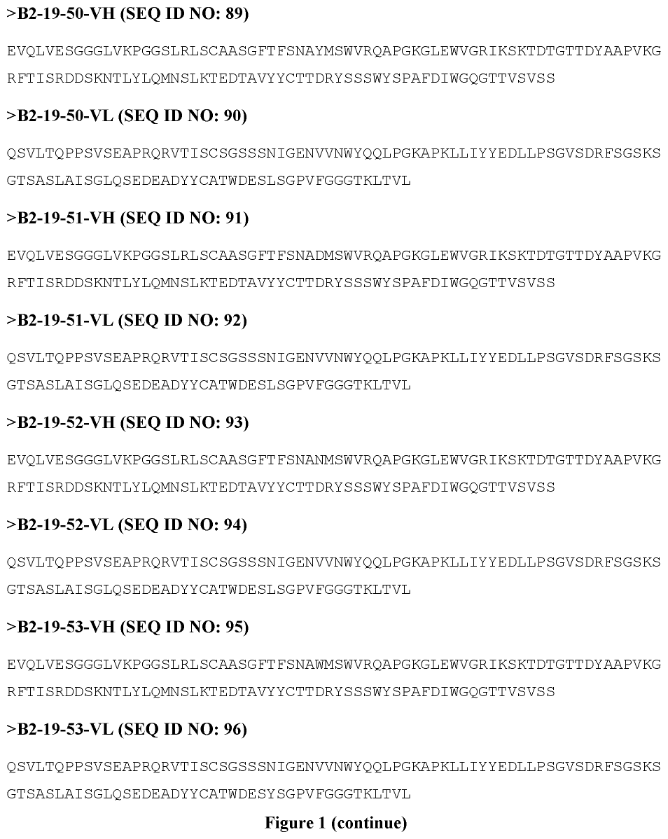

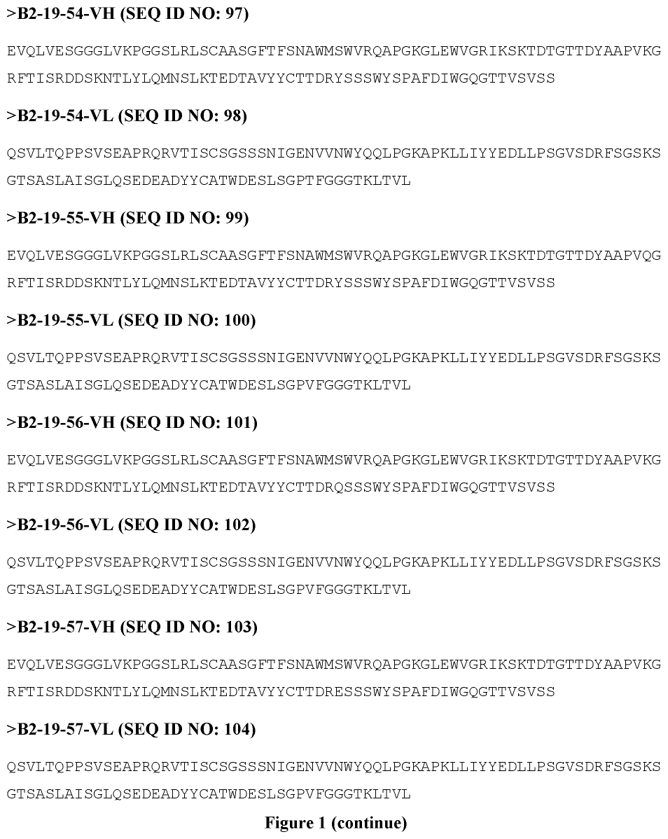

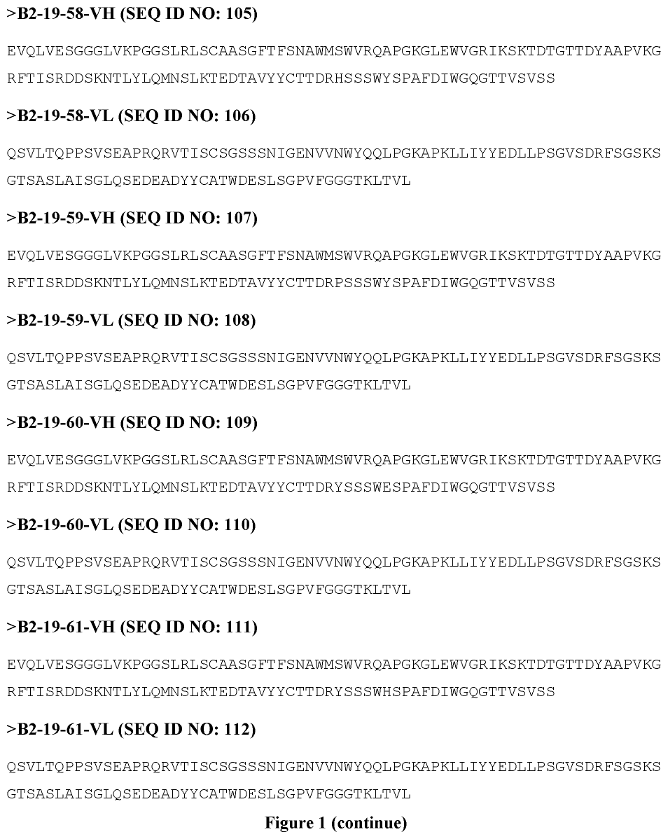

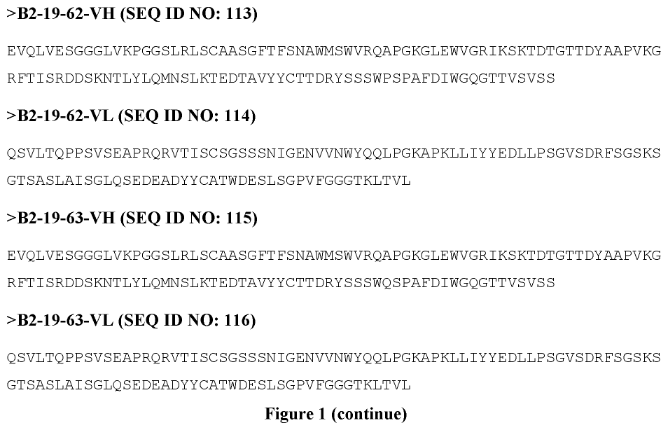

In certain embodiments, the LILRB2 antibodies disclosed herein have a clone-paired heavy chain variable region (VH) and light chain variable region (VL) amino acid sequences as provided in FIG. 1.

In some embodiments, the antibody or antigen-binding fragment thereof comprises the heavy chain variable region has an amino acid sequence of SEQ ID NO: 25 and the light chain variable region has an amino acid sequence of SEQ ID NO: 26. In some embodiments, the antibody or antigen-binding fragment thereof comprises the heavy chain variable region has an amino acid sequence of SEQ ID NO: 31 and the light chain variable region has an amino acid sequence of SEQ ID NO: 32.

In one embodiment, the anti-LILRB2 antibodies and the antigen-binding fragments provided herein is a single domain antibody which consists of all or a portion of the heavy chain variable domain provided herein. More information of such a single domain antibody is available in the art (see, e.g., U.S. Pat. No. 6,248,516).

In certain embodiments, the anti-LILRB2 antibodies and the fragments thereof provided herein further comprise an immunoglobulin constant region. In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises Cκ or Cλ.

The antibodies or antigen-binding fragments thereof provided herein can be a monoclonal antibody, polyclonal antibody, recombinant antibody, bispecific antibody, labeled antibody, bivalent antibody, or anti-idiotypic antibody. A recombinant antibody is an antibody prepared in vitro using recombinant methods rather than in animals.

### Antibody Variants

The antibodies and antigen-binding fragments thereof provided herein also encompass various variants thereof. In certain embodiments, the antibodies and antigen-binding fragments thereof encompasses various types of variants of an exemplary antibody provided herein.

In certain embodiments, the antibody variants comprise one or more modifications or substitutions in one or more variable region sequences provided herein, and/or the constant region (e.g., Fc region). Such variants retain specific binding affinity to LILRB2 and ligand blocking ability of their parent antibodies but have one or more desirable properties conferred by the modification(s) or substitution(s). For example, the antibody variants may have improved antigen-binding affinity, improved glycosylation pattern, reduced risk of glycosylation, reduced deamidation or deamination, improved or increased effector function(s), reduced or depleted effector function(s), improved FcRn receptor binding, increased pharmacokinetic half-life, pH sensitivity, reduced immunogenicity and/or compatibility to conjugation (e.g. one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

### Affinity Variant

Affinity variant may contain modifications or substitutions in the heavy or light chain variable region sequences provided herein. The affinity variants retain specific binding affinity to LILRB2 of the parent antibody, or even have improved LILRB2 specific binding affinity over the parent antibody.

Various methods known in the art can be used to achieve this purpose. For example, a library of antibody variants (such as Fab or scFv variants) can be generated and expressed with phage display technology, and then screened for the binding affinity to LILRB2. For another example, computer software can be used to virtually simulate the binding of the antibodies to LILRB2 and identify the amino acid residues on the antibodies which form the binding interface. Such residues may be either avoided in the substitution so as to prevent reduction in binding affinity or targeted for substitution to provide for a stronger binding.

In certain embodiments, the antibody or antigen-binding fragment provided herein comprises one or more amino acid residue substitutions in one or more CDR sequences, and/or one or more FR sequences. In certain embodiments, an affinity variant comprises no more than 10, 9, 8, 7, 6, 5, 4, 3, 2, or 1 substitution in the CDR sequences and/or FR sequences in total.

In certain embodiments, the anti-LILRB2 antibodies and antigen-binding fragments thereof comprise one or more variable region sequences having at least 80% (e.g. at least 85%, 88%, 90%, 91%, 92%, 93%, 94%, 95%, 96%, 97%, 98%, 99%) sequence identity to that (or those) provided herein, and in the meantime retain the binding affinity to LILRB2 at a level similar to or even higher than its parent antibody. In some embodiments, the substitutions, insertions, or deletions occur in the CDR regions. In some embodiments, the substitutions, insertions, or deletions occur in regions outside the CDRs (e.g., in the FRs).

### Glycosylation Variant

The anti-LILRB2 antibodies and antigen-binding fragments provided herein also encompass a glycosylation variant, which can be obtained to either increase or decrease the extent of glycosylation of the antibody or antigen binding fragment.

In some embodiment, the anti-LILRB2 antibodies and antigen-binding fragments provided herein comprises a particular glycosylation pattern. For example, an aglycosylated antibody can be made (i.e., the antibody lacks glycosylation). The glycosylation pattern of an antibody may be altered to, for example, increase the affinity or avidity of the antibody for an antigen. Such modifications can be accomplished by, for example, altering one or more of the glycosylation sites within the antibody sequence. For example, one or more amino acid substitutions can be made that result removal of one or more of the variable region framework glycosylation sites to thereby eliminate glycosylation at that site. Such aglycosylation may increase the affinity or avidity of the antibody for antigen. See, e.g., U.S. Pat. Nos. 5,714,350 and 6,350,861.

An antibody may also be made in which the glycosylation pattern includes hypofucosylated or afucosylated glycans, such as a hypofucosylated antibodies or afucosylated antibodies have reduced amounts of fucosyl residues on the glycan. The antibodies may also include glycans having an increased amount of bisecting GlcNac structures. Such altered glycosylation patterns have been demonstrated to increase the ADCC ability of antibodies. Such modifications can be accomplished by, for example, expressing the antibodies in a host cell in which the glycosylation pathway was been genetically engineered to produce glycoproteins with particular glycosylation patterns. These cells have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (a (1,6)-fucosyltransferase), such that antibodies expressed in the Ms704, Ms705, and Ms709 cell lines lack fucose on their carbohydrates. The Ms704, Ms705, and Ms709 FUT8-/- cell lines were created by the targeted disruption of the FUT8 gene in CHO/DG44 cells using two replacement vectors (see U.S. Patent Publication No. 20040110704. As another example, EP 1 176 195 describes a cell line with a functionally disrupted FUT8 gene, which encodes a fucosyl transferase, such that antibodies expressed in such a cell line exhibit hypofucosylation by reducing or eliminating the α-1,6 bond-related enzyme. EP 1 176 195 also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 2003/035835 describes a variant CHO cell line, Lec13 cells, with reduced ability to attach fucose to Asn(297)-linked carbohydrates, also resulting in hypofucosylation of antibodies expressed in that host cell. Antibodies with a modified glycosylation profile can also be produced in chicken eggs, as described in PCT Publication WO 06/089231. Alternatively, antibodies with a modified glycosylation profile can be produced in plant cells, such as Lemna (U.S. Pat. No. 7,632,983). Methods for production of antibodies in a plant system are disclosed in the U.S. Pat. Nos. 6,998,267 and 7,388,081. PCT Publication WO1999/054342 describes cell lines engineered to express glycoprotein-modifying glycosyl transferases (e.g., β(1,4)-N-acetylglucosaminyltransferase III (GnTIII)) such that antibodies expressed in the engineered cell lines exhibit increased bisecting GlcNac structures which results in increased ADCC activity of the antibodies. Hypofucosylation is also called afucosylation when fucosylation is minimal on antibodies.

Alternatively, the fucose residues of the antibodies can be cleaved off using a fucosidase enzyme; e.g., the fucosidase α-L-fucosidase removes fucosyl residues from antibodies. Antibodies disclosed herein further include those produced in lower eukaryote host cells, in particular fungal host cells such as yeast and filamentous fungi have been genetically engineered to produce glycoproteins that have mammalian- or human-like glycosylation patterns. A particular advantage of these genetically modified host cells over currently used mammalian cell lines is the ability to control the glycosylation profile of glycoproteins that are produced in the cells such that compositions of glycoproteins can be produced wherein a particular N-glycan structure predominates (see, e.g., U.S. Pat. Nos. 7,029,872 and 7,449,308). These genetically modified host cells have been used to produce antibodies that have predominantly particular N-glycan structures.

In addition, since fungi such as yeast or filamentous fungi lack the ability to produce fucosylated glycoproteins, antibodies produced in such cells will lack fucose unless the cells are further modified to include the enzymatic pathway for producing fucosylated glycoproteins (See for example, PCT Publication WO2008112092). In particular embodiments, the antibodies disclosed herein further include those produced in lower eukaryotic host cells and which comprise fucosylated and nonfucosylated hybrid and complex N-glycans, including bisected and multiantennary species, including but not limited to N-glycans such as GlcNAc(1-4)Man3G1cNAc2; Gal(1-4)G1cNAc(1-4)Man3G1cNAc2; NANA(1-4)Gal(1-4)G1cNAc(1-4)Man3G1cNAc2. In particular embodiments, the antibody compositions provided herein may comprise antibodies having at least one hybrid N-glycan selected from the group consisting of GlcNAcMan5G1cNAc2; GalG1cNAcMan5G1cNAc2; and NANAGalG1cNAcMan5G1cNAc2. In particular aspects, the hybrid N-glycan is the predominant N-glycan species in the composition. In further aspects, the hybrid N-glycan is a particular N-glycan species that comprises about 30%, 40%, 50%, 60%, 70%, 80%, 90%, 95%, 97%, 98%, 99%, or 100% of the hybrid N-glycans in the composition.

In particular embodiments, the antibody compositions provided herein comprise antibodies having at least one complex N-glycan selected from the group consisting of GlcNAcMan3 GlcNAc2; GalG1cNAcMan3G1cNAc2; NANAGal GlcNAcMan3 GlcNAc2; GlcNAc2Man3 GlcNAc2; Gal GlcNAc2Man3 GlcNAc2; Gal2G1cNAc2Man3G1cNAc2; NANAGal2G1cNAc2Man3G1cNAc2; and NANA2Gal2G1cNAc2Man3G1cNAc2. In particular aspects, the complex N-glycan is the predominant N-glycan species in the composition. In further aspects, the complex N-glycan is a particular N-glycan species that comprises about 30%, 40%, 50%, 60%, 70%, 80%, 90%, 95%, 97%, 98%, 99%, or 100% of the complex N-glycans in the composition. In particular embodiments, the N-glycan is fusosylated. In general, the fucose is in an α1,3-linkage with the GlcNAc at the reducing end of the N-glycan, an α1,6-linkage with the GlcNAc at the reducing end of the N-glycan, an α1,2-linkage with the Gal at the non-reducing end of the N-glycan, an α1,3-linkage with the GlcNac at the non-reducing end of the N-glycan, or an α1,4-linkage with a GlcNAc at the non-reducing end of the N-glycan.

Therefore, in particular aspects of the above the glycoprotein compositions, the glycoform is in an α1,3-linkage or α1,6-linkage fucose to produce a glycoform selected from the group consisting of Man5GlcNAc2(Fuc), GlcNAcMan5GlcNAc2(Fuc), Man3G1cNAc2(Fuc), GlcNAcMan3G1cNAc2(Fuc), GlcNAc2Man3G1cNAc2(Fuc), GalG1cNAc2Man3 G1cNAc2(Fuc), Gal2G1cNAc2Man3 G1cNAc2(Fuc), NANAGal2G1cNAc2Man3G1cNAc2(Fuc), and NANA2Gal2G1cNAc2Man3G1cNAc2(Fuc); in an α1,3-linkage or α1,4-linkage fucose to produce a glycoform selected from the group consisting of GlcNAc(Fuc)Man5G1cNAc2, GlcNAc(Fuc)Man3G1cNAc2, GlcNAc2(Fuc1-2)Man3 GlcNAc2, Gal GlcNAc2(Fuc1-2)Man3 GlcNAc2, Gal2GlcNAc2(Fuc1-2)Man3 GlcNAc2, NANAGal2 GlcNAc2(Fuc1-2)Man3 GlcNAc2, and NANA2Gal2G1cNAc2(Fuc1-2)Man3G1cNAc2; or in an α1,2-linkage fucose to produce a glycoform selected from the group consisting of Gal(Fuc)G1cNAc2Man3G1cNAc2, Gal2(Fuc1-2)G1cNAc2Man3 GlcNAc2, NANAGal2(Fuc1-2)G1cNAc2Man3 GlcNAc2, and NANA2Gal2(Fuc1-2)G1cNAc2Man3 GlcNAc2.

In further aspects, the antibodies comprise high mannose N-glycans, including but not limited to, Man8GlcNAc2, Man7GlcNAc2, Man6GlcNAc2, Man5GlcNAc2, Man4GlcNAc2, or N-glycans that consist of the Man3GlcNAc2 N-glycan structure. In further aspects of the above, the complex N-glycans further include fucosylated and non-fucosylated (or afucosylated) bisected and multiantennary species. As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein.

### Cysteine-Engineered Variant

The anti-LILRB2 antibodies and antigen-binding fragments provided herein also encompass a cysteine-engineered variant, which comprises one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments to introduce free cysteine residues are known in the art, see, for example, WO2006/034488.

### Fc Variant

The anti-LILRB2 antibodies and antigen-binding fragments disclosed herein can also be engineered to include modifications within the Fc region, typically to alter one or more functional properties of the antibody, such as serum half-life, complement fixation, Fc receptor binding, and/or effector function (e.g., antigen-dependent cellular cytotoxicity). Furthermore, the antibodies disclosed herein can be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat. The antibodies disclosed herein also include antibodies with modified (or blocked) Fc regions to provide altered effector functions. See, e.g., U.S. Pat. No. 5,624,821; WO2003/086310; US2004/0002587; US2005/0152894; US2005/0249723; WO2006/019447. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, enabling less frequent dosing and thus increased convenience and decreased use of material. This mutation has been reported to abolish the heterogeneity of inter-heavy chain disulfide bridges in the hinge region.

In one embodiment, the hinge region of CH1 is modified such that the number of cysteine residues in the hinge region is increased or decreased. This approach is described further in U.S. Pat. No. 5,677,425. The number of cysteine residues in the hinge region of CH1 is altered, for example, to facilitate assembly of the light and heavy chains or to increase or decrease the stability of the antibody. In another embodiment, the antibody is modified to increase its biological half-life. Various approaches are possible. For example, one or more of the following mutations can be introduced: T252L, T254S, T256F, as described in U.S. Pat. No. 6,277,375. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022. In yet other embodiments, the Fc region is altered by replacing at least one amino acid residue with a different amino acid residue to alter the effector function(s) of the antibodies. For example, one or more amino acids selected from amino acid residues 234, 235, 236, 237, 297, 318, 320 and 322 can be replaced with a different amino acid residue such that the antibody has an altered affinity for an effector ligand but retains the antigen binding ability of the parent antibody. The effector ligand to which affinity is altered can be, for example, an Fc receptor or the C1 component of complement. This approach is described in further detail in U.S. Pat. Nos. 5,624,821 and 5,648,260.

In another example, one or more amino acid residues within amino acid positions 231 and 239 are altered to thereby alter the ability of the antibody to fix complement. This approach is described further in PCT Publication WO1994/029351. In yet another example, the Fc region is modified to increase or decrease the ability of the antibodies to mediate antibody dependent cellular cytotoxicity (ADCC) and/or to increase or decrease the affinity of the antibodies for an Fcγ receptor by modifying one or more amino acids at the following positions: 238, 239, 243, 248, 249, 252, 254, 255, 256, 258, 264, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 2000/042072. Moreover, the binding sites on human IgG1 for FcγR1, FcγRII, FcγRIII and FcRn have been mapped and variants with improved binding have been described. Specific mutations at positions 256, 290, 298, 333, 334 and 339 were shown to improve binding to FcγRIII Additionally, the following combination mutants were shown to improve FcγRIII binding: T256A/S298A, S298A/E333A, S298A/K224A and S298A/E333A/K334A.

In one embodiment, the Fc region is modified to decrease the ability of the antibodies to mediate effector function and/or to increase anti-inflammatory properties by modifying residues 243 and 264. In one embodiment, the Fc region of the antibody is modified by changing the residues at positions 243 and 264 to alanine. In one embodiment, the Fc region is modified to decrease the ability of the antibody to mediate effector function and/or to increase anti-inflammatory properties by modifying residues 243, 264, 267 and 328.

In one embodiment, the Fc region is modified to abolish the ability of the antibodies to mediate effector function by modifying residues 234, 235 and 329 to alanine or glycine (L234A-L235A-P329G).

In certain embodiments, the anti-LILRB2 antibodies or antigen-binding fragments comprise one or more amino acid substitution(s) that improves the pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al., Structure, 6(1): 63-73, 1998; Kontermann, R. et al., Antibody Engineering, Volume 1, Chapter 27: Engineering of the Fc region for improved PK, published by Springer, 2010; Yeung, Y. et al., Cancer Research (2010) 70: 3269-3277; and Hinton, P. et al., J. Immunology (2006) 176:346-356.

In certain embodiments, the anti-LILRB2 antibodies or antigen-binding fragments comprise one or more amino acid substitution(s) that alters the antibody-dependent cellular cytotoxicity (ADCC). Certain amino acid residues at CH2 domain of the Fc region can be substituted to provide for enhanced ADCC activity. Alternatively, or additionally, carbohydrate structures on the antibody can be changed to enhance ADCC activity. Methods of altering ADCC activity by antibody engineering have been described in the art, see for example, Shields R L. et al., J Biol Chem. (2001) 276(9): 6591-604; Idusogie E E. et al., J Immunol. (2000) 164(8):4178-84; Steurer W. et al., J Immunol. (1995) 155(3): 1165-74; Idusogie E E. et al., J Immunol. (2001) 166(4): 2571-5; Lazar G A. et al., PNAS (2006) 103(11): 4005-4010; Ryan M C. et al., Mol. Cancer Ther. (2007) 6: 3009-3018; Richards J O. et al., Mol Cancer Ther. (2008) 7(8): 2517-27; Shields R. L. et al., J. Biol. Chem, 2002, 277: 26733-26740; Shinkawa T. et al., J. Biol. Chem (2003) 278: 3466-3473.

In certain embodiments, the anti-LILRB2 antibodies or antigen-binding fragments comprise one or more amino acid substitution(s) that alters Complement Dependent Cytotoxicity (CDC), for example, by improving or diminishing C1q binding and/or CDC (see, for example, WO99/51642; Duncan & Winter Nature 322:738-40 (1988); U.S. Pat. Nos. 5,648,260; 5,624,821); and WO1994/029351 concerning other examples of Fe region variants.

In certain embodiments, the anti-LILRB2 antibodies or antigen-binding fragments comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

### Antigen-Binding Fragments

Various types of antigen-binding fragments are known in the art and can be developed based on the anti-LILRB2 antibodies provided herein, including for example, the exemplary antibodies whose variable sequences are provided herein, and their different variants (such as affinity variants, glycosylation variants, Fc variants, cysteine-engineered variants and so on).

In certain embodiments, an anti-LILRB2 antigen-binding fragment provided herein is a camelized single domain antibody, a diabody, a single chain Fv fragment (scFv), an scFv dimer, a BsFv, a dsFv, a (dsFv)₂, a dsFv-dsFv′, an Fv fragment, a Fab, a Fab′, a F(ab′)₂, a ds-diabody, a nanobody, a domain antibody, a single domain antibody, or a bivalent domain antibody.

A Single Chain Variable Fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alaine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the VH C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., Nhydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338 describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

Various techniques can be used for the production of such antigen-binding fragments. Illustrative methods include, enzymatic digestion of intact antibodies (see, e.g., Morimoto et al., Journal of Biochemical and Biophysical Methods (1992) 24:107-117; and Brennan et al., Science (1985) 229:81), recombinant expression by host cells such as E. Coli (e.g. for Fab, Fv and ScFv antibody fragments), screening from a phase display library as discussed above (e.g. for ScFv), and chemical coupling of two Fab′-SH fragments to form F(ab′)₂ fragments (Carter et al., Bio/Technology (1992) 10:163-167). Other techniques for the production of antibody fragments will be apparent to a skilled practitioner.

In certain embodiments, the antigen-binding fragment is a scFv. Generation of scFv is described in, for example, WO 93/16185; U.S. Pat. Nos. 5,571,894; and 5,587,458. scFv may be fused to an effector protein at either the amino or the carboxyl terminus to provide for a fusion protein (see, for example, Antibody Engineering, ed. Borrebaeck).

### Multi-Specific Antibodies

In certain embodiments, the anti-LILRB2 antibodies and antigen-binding fragments thereof provided herein are multi-specific. The term “multi-specific” as used herein encompasses molecules having more than one specificity, e.g., bispecific, tri-specific, tetra-specific. In certain embodiments, the multi-specific antibodies and antigen-binding fragments thereof provided herein are capable of specifically binding to a first and a second epitopes of LILRB2, while the first epitope and the second epitopes of LILRB2 are distinct from each other or non-overlapping. In certain embodiments, the multi-specific antibodies and antigen-binding fragments thereof provided herein is capable of specifically binding to LILRB2 and a second antigen different from LILRB2.

In certain embodiments, the second antigen is an immune related target. An immune related target as used herein, encompasses a biological molecule that is involved in the stimulation, inhibition or modulation of an immune response, optionally, cellular immune responses. An example of the immune related target is an immune modulator molecule that is expressed by a cancer cell, a stromal cell (fibroblast, vascular cell, etc.) or an immune cell. In some embodiments, the immune modulator molecule can mediate co-stimulatory signal to augment immune response or can mediate co-inhibitory signals to suppress immune response. Therefore, in some embodiment, the second antigen is an immune modulator molecule.

In some embodiment, the immune modulator molecule is PD-1, PD-L1, PD-L2, CTLA-4, LAG3, TIM-3, Fc receptors, FCRL(1-6), A2AR, CD160, 2B4, TGF-β, TGF-βR, VISTA, BTLA, TIGIT, LAIR1, LILRB1, LILRB3, LILRB4, LILRB5, LILRA(1-6), OX40, CD2, CD27, CD28, CD30, CD40, CD47, SIRPA, CLEC-1, clever-1/stabilin-1, ADGRE, TREM1, TREM2, CD122, ICAM-1, IDO, NKG2D/C, SLAMF7, MS4A4A, SIGLEC(7-15), NKp80, NKG2A, CD160, CD161, CD300, CD163, B7-H3, B7-H4, LFA-1, ICOS, 4-1BB, GITR, BAFFR, HVEM, CD7, LIGHT, TNFR2, TLR(1-9), IL-2, IL-7, IL-15, IL-21, CD16 and CD83.

In certain embodiments, the second antigen comprises a tumor antigen. “Tumor antigen” as used herein refers to tumor specific antigens (e.g., those unique to tumor cells and normally not found on non-tumor cells), and tumor-associated antigens (e.g., found in both tumor and non-tumor cells but expressed differently in tumor cells, or found in tumor microenvironment). Tumor specific antigens can also include tumor neo-antigens (e.g., that are expressed in cancer cells because of somatic mutations that change the protein sequence or create fusion proteins between two unrelated sequences).

Examples of tumor antigens include, without limitation, prostate specific antigen (PSA), CA-125, gangliosides G(D2), G(M2) and G(D3), CD20, CD52, CD33, Ep-CAM, CEA, bombesin-like peptides, HER2/neu, epidermal growth factor receptor (EGFR), erbB2, erbB3/HER3, erbB4, FGFR2b, CD44v6, cancer-associated mucin, VEGF, VEGFRs (e.g., VEGFR3), estrogen receptors, Lewis-Y antigen, TGFβ1, IGF-1 receptor, EGFα, c-Kit receptor, transferrin receptor, Claudin 18.2, GPC-3, Nectin-4, ROR1, methothelin, BCMA, MAGE-1, MAGE-3, BAGE, GAGE-1, GAGE-2, p15, BCR-ABL, E2APRL, H4-RET, IGH-IGK, MYL-RAR, IL-2R, C017-1A, TROP2, Ephrin A, or LIV-1.

Multi-specific antibodies and antigen-binding fragments thereof provided herein can be in a suitable format known in the art. For example, an exemplary bispecific format can be bispecific diabodies, scFv-based bispecific formats, IgG-scFv fusions, dual variable domain (DVD)-Ig, Quadroma, knobs-into-holes, common light chain (e.g., common light chain with knobs-into-holes, etc.), BiTE, CrossMab, CrossFab, Duobody, SEEDbody, leucine zipper, dual acting Fab (DAF)-IgG, and Mab e bispecific formats (see, e.g., Brinkmann et al. 2017, Mabs, 9(2): 182-212). The bispecific molecules can be in symmetric or asymmetric architecture.

The multi-specific antibodies and antigen-binding fragments provided herein can be made with any suitable methods known in the art. In one embodiment, two immunoglobulin heavy chain-light chain pairs having different antigenic specificities are co-expressed in a host cell to produce bispecific antibodies in a recombinant way (see, for example, Milstein and Cuello, Nature, 305: 537 (1983)), followed by purification by affinity chromatography.

### Conjugates

In some embodiments, the anti-LILRB2 antibodies and antigen-binding fragments thereof further comprise a conjugate moiety. The conjugate moiety can be linked to the antibodies and antigen-binding fragments thereof. A conjugate moiety is a proteinaceous or non-proteinaceous moiety that can be attached to the antibody or antigen-binding fragment thereof. It is contemplated that a variety of conjugate moieties may be linked to the antibodies or antigen-binding fragments provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments by covalent binding, affinity binding, intercalation, coordinate binding, complexation, association, blending, or addition, among other methods.

In certain embodiments, the antibodies and antigen-binding fragments disclosed herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the antibodies may be linked to a conjugate moiety indirectly, or through another conjugate moiety. For example, the antibody or antigen-binding fragments may be conjugated to biotin, then indirectly conjugated to a second conjugate that is conjugated to avidin.

Examples of conjugate moiety include without limitation an immune modulatory agent, an anti-tumor drug, a STING (Stimulator of Interferon Genes) agonist, a cytokine, a clearance-modifying agent, a toxin (e.g., a chemotherapeutic agent), an immune cell stimulator (e.g., a TLR agonist), a detectable label (e.g., a radioactive isotope, a lanthanide, a luminescent label, a fluorescent label, or an enzyme-substrate label), a DNA, an RNA, or purification moiety.

Examples of immune modulatory agent include without limitation an immune modulator molecule disclosed herein (e.g., PD-1, PD-L1, PD-L2, CTLA-4, LAG3, TIM-3, Fc receptors, FCRL(1-6), A2AR, CD160, 2B4, TGF-β, TGF-βR, VISTA, BTLA, TIGIT, LAIR1, LILRB1, LILRB3, LILRB4, LILRB5, LILRA(1-6), OX40, CD2, CD27, CD28, CD30, CD40, CD47, SIRPA, CLEC-1, clever-1/stabilin-1, ADGRE, TREM1, TREM2, CD122, ICAM-1, IDO, NKG2D/C, SLAMF7, MS4A4A, SIGLEC(7-15), NKp80, NKG2A, CD160, CD161, CD300, CD163, B7-H3, B7-H4, LFA-1, ICOS, 4-1BB, GITR, BAFFR, HVEM, CD7, LIGHT, TNFR2, TLR(1-9), IL-2, IL-7, IL-15, IL-21, CD16 and CD83), or a functional fragment thereof, a ligand thereof, and a ligand-binding protein thereof.

Examples of anti-tumor drugs include without limitation a chemotherapeutic agent, a growth inhibitory agent, a cytotoxic agent, an agent used in radiation therapy, an anti-angiogenesis agent, a cancer immunotherapeutic agent, a apoptotic agent, an anti-tubulin agent, an anti-HER-2 antibody, an anti-CD20 antibody, an epidermal growth factor receptor (EGFR) antagonist, HER1/EGFR inhibitor, a platelet derived growth factor inhibitor, a COX-2 inhibitor, an interferon, a CTLA4 inhibitor (e.g., anti-CTLA antibody ipilimumab (YERVOY®), or tremelimumab), a PD-1 or PD-L1 inhibitor (e.g., OPDIVO® or nivolumab, KEYTRUDA® or pembrolizumab, TECENTRIQ® or atezolizumab, BAVENCIO® or avelumab, IMFINZI® or durvalumab, LIBTAYO® or cemiplimab-rwlc, TYVYT® or sintilimab, tislelizumab (BGB-A317), penpulimab (AK105), camrelizumab, toripalimab, zimberelimab (GLS-010), retifanlimab, sugemalimab, or CS1003), a dual-targeting antibody against CTLA-4 and PD-1 or PD-L1 (e.g., an anti-PD-1/CTLA-4 bi-specific antibody or AK104), a TIM3 inhibitor (e.g., anti-TIM3 antibodies), a LAG-3 inhibitor (e.g., anti-LAG3 antibodies), a cytokine, an antagonist (e.g., neutralizing antibodies) that bind to one or more of the following targets ErbB2, ErbB3, ErbB4, FGFR2b, PDGFR-beta, BlyS, APRIL, BCMA, or VEGF receptor(s), TRAIL/Apo2, an IDH1 inhibitor, an ivosidenib, Tibsovo®, an IDH2 inhibitor, an enasidenib, Idhifa®, a smoothened (SMO) inhibitor, a glasdegib, an arginase inhibitor, an IDO inhibitor, an epacadostat, a BCL-2 inihbitor, a venetoclax, Venclexta®, a platinum complex derivative, oxaliplatin, a kinase inhibitor, a tyrosine kinase inhibitor, a PI3 kinase inhibitor, a BTK inhibitor, an ibrutinib, IMBRUVICA®, an acalabrutinib, CALQUENCE®, a zanubrutinib, a TLR agonist, a STING agonist, an ICOS antibody, a TIGIT antibody, a CD40 antibody, a 4-1BB antibody, a CD47 antibody, an OX40 antibody, a TNFR2 antibody, an antibody to another LILR family member, a Siglec antibody, a SIRP la antibody or fusions protein, an antagonist of E-selectin, an antibody binding to a tumor antigen, an antibody binding to a T cell surface marker, an antibody binding to a myeloid cell or NK cell surface marker, an alkylating agent, a nitrosourea agent, an antimetabolite, an antitumor antibiotic, an alkaloid derived from a plant, a hormone therapy medicine, a hormone antagonist, an aromatase inhibitor, and a P-glycoprotein inhibitor, an engineered T cell, NK cell or macrophage.

A “toxin” can be any agent that is detrimental to cells or that can damage or kill cells. Examples of toxin include, without limitation, taxol, deruxtecan, cytochalasin B, gramicidin D, ethidium bromide, emetine, mitomycin, etoposide, tenoposide, vincristine, monomethyl auristatin E (MMAE), monomethyl auristatin F (MMAF), mertansine, emtansine, DM1, maytansinoid DM1, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), a topoisomerase inhibitor, and a tubulin-binders.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or P-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁵Cu, ⁶⁷CU, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), luminescent labels, chromophoric moiety, digoxigenin, biotin/avidin, a DNA molecule or gold for detection.

In certain embodiments, the conjugate moiety can be a clearance-modifying agent which helps increase half-life of the antibody. Illustrative examples include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymers are attached, they can be the same or different molecules.

In certain embodiments, the conjugate moiety can be a purification moiety such as a magnetic bead.

In certain embodiments, the antibodies and antigen-binding fragments thereof provided herein is used for a base for a conjugate.

### Polynucleotides and Recombinant Methods

The present disclosure provides isolated polynucleotides that encode the anti-LILRB2 antibodies and antigen-binding fragments thereof. In certain embodiments, the isolated polynucleotides comprise one or more nucleotide sequences that encode the variable region of the exemplary antibodies provided herein. DNA encoding the monoclonal antibody is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the anti-LILRB2 antibodies and antigen-binding fragments can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

The present disclosure provides vectors (e.g., expression vectors) containing the nucleic acid sequence provided herein encoding the antibodies or antigen-binding fragments, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-LILRB2 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment provided here are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. (1977) 36:59); baby hamster kidney cells (BHK, ATCC CCL 10); Chinese Hamster Ovary cells (CHO), CHO cells deficient in dihydrofolate reductase (DHFR) activity, CHO-DHFR (Urlaub et al., Proc. Natl. Acad. Sci. USA (1980) 77:4216); mouse sertoli cells (TM4, Mather, Biol. Reprod. (1980) 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. (1982) 383:44-68); MRC 5 cells; FS4 cells; and a human hepatoma line (Hep G2). In some preferable embodiments, the host cell is 293F cell.

Host cells are transfected with the above-described expression or cloning vectors for anti-LILRB2 antibody production and cultured in conventional nutrient media modified as appropriate for inducing promoters, selecting transformants, or amplifying the genes encoding the desired sequences. In another embodiment, the antibody may be produced by homologous recombination known in the art.

The host cells used to produce the antibodies or antigen-binding fragments provided herein may be cultured in a variety of media. Commercially available media such as Ham's F10 (Sigma), Minimal Essential Medium (MEM) (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. (1980) 102:255, U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant DNA techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology (1992) 10:163-167 describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-LILRB2 antibodies and antigen-binding fragments prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. (1983) 62:1-13). Protein G is recommended for all mouse isotypes and for human gamma3 (Guss et al., EMBO J. (1986)5:1567-75). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE' chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS-PAGE (Capaldi et al., 1977). It will therefore be appreciated that under differing electrophoresis conditions, the apparent molecular weights of purified or partially purified expression products may vary.

### III. Pharmaceutical Composition

The present disclosure further provides pharmaceutical compositions comprising the anti-LILRB2 antibodies or antigen-binding fragments thereof and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment and conjugates as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibodies or antigen-binding fragments as disclosed herein and one or more antioxidants such as methionine. Further provided are methods for preventing oxidation of, extending the shelf-life of, and/or improving the efficacy of an antibody or antigen-binding fragment as provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbitol, fructose, corn syrup, xylitol, glycerin, glucose, sucrose or other suitable agents. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-LILRB2 antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given and can be empirically determined.

In certain embodiments, the pharmaceutical compositions comprising the anti-LILRB2 antibodies or antigen-binding fragments thereof described herein further comprise one or more additional therapeutic agents that are co-administered with the anti-LILRB2 antibodies or antigen-binding fragments thereof. The candidates of the additional therapeutic agents are disclosed infra in Section IV. It can be understood that the additional therapeutic agents can be co-formulated with the anti-LILRB2 antibodies or antigen-binding fragments thereof, or be mixed with the anti-LILRB2 antibodies or antigen-binding fragments thereof right before the administration, such as in the IV infusion bag.

### IV. Methods of Use of Anti-LILRB2 Antibodies

LILRB2 has been identified as a key regulator of myeloid cell phenotype. The activation of LILRB2 suppresses the pro-inflammatory activity of myeloid cells. While myeloid cells with a suppressive/anti-inflammatory phenotype can down-regulate the activation, proliferation and cytotoxic activity of T cells, modulation of LILRB2 has the potential in therapeutic use in conditions and disorders including cancer, infectious diseases (e.g., chronic viral infection disease), autoimmune diseases, and inflammatory diseases.

Therefore, the present disclosure also provides therapeutic methods using the anti-LILRB2 antibody or antigen-binding fragment as provided herein. In some embodiments the method comprises: administering a therapeutically effective amount of the antibody or antigen-binding fragment as provided herein to a subject in need thereof, thereby treating or preventing a LILRB2-related condition or disorder. In some embodiment, the LILRB2-related condition or disorder is cancer, infectious disease, autoimmune disease and inflammatory disease.

Examples of cancer can be generally categorized into solid tumors and hematologic malignancies. Solid tumors include but are not limited to, non-small cell lung cancer (squamous/non-squamous), small cell lung cancer, renal cell cancer, colorectal cancer, colon cancer, ovarian cancer, breast cancer (including basal breast carcinoma, ductal carcinoma and lobular breast carcinoma), pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, melanoma, multiple myeloma, mycoses fungoides, Merkel cell cancer, hepatocellular carcinoma (HCC), fibrosarcoma, myxosarcoma, liposarcoma, chondrosarcoma, osteogenic sarcoma, and other sarcomas, synovioma/synovial sarcoma, mesothelioma, Ewing's sarcoma, leiomyosarcoma, rhabdomyosarcoma, basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinomas, medullary carcinoma, bronchogenic carcinoma, hepatoma, bile duct carcinoma, choriocarcinoma, Wilms' tumor, cervical cancer, testicular tumor, seminoma, mast cell derived tumors, EBV-positive and -negative PTLD, nasopharyngeal carcinoma, spinal axis tumor, brain stem glioma, astrocytoma, medulloblastoma, craniopharyogioma, ependymoma, pinealoma, hemangioblastoma, acoustic neuroma, oligodendroglioma, meningioma, melanoma, neuroblastoma and retinoblastoma.

Solid tumors are characterized by multiple biologic hallmarks including sustaining proliferative signaling, evading growth suppressors, resisting cell death, enabling replicative immortality, inducing angiogenesis, activating invasion and metastasis, tumor promoting inflammation, avoiding immune destruction, genomic instability and mutation, and deregulating cellular energetics. Treatment efforts have evolved from cytotoxic chemotherapies targeting rapidly dividing cells to small molecules inhibiting select signaling pathways, to monoclonal antibodies targeting cell surface proteins. More recently, the concept of cancer immunotherapy, either through reinvigoration of endogenous anti-tumor immunity or via cellular therapies utilizing synthetic immunity, has shown promise. Despite these advances, most patients with advanced solid tumors still do not survive long-term. The use of immune checkpoint inhibitors such as anti-CTLA-4 or anti-PD-1/PD-L1 have led to long-term progression-free and overall survival in a minority of patients.

Newer immunotherapy approaches targeting different aspects of immune biology and different tumor-infiltrating cells are needed to improve outcomes, such as those targeting LILRB2 as an inhibitory receptor expressed on myeloid cells, including myeloid-derived suppressor cells (MDSC), tolerogenic DCs and tumor-associated macrophages (TAMs). These myeloid cells are described functionally as immune suppressive cells because their immune suppressive/anti-inflammatory phenotype can inhibit the activation, proliferation and cytotoxic activity of tumor antigen-specific T cells.

In some embodiments, depleting immune suppressive cells may revert the suppressive effect on tumor antigen-specific T cells for solid tumor treatment.

In some embodiments, blocking LILRB2 on myeloid cells could also unlock its inhibitory effects on antigen presentation cells (APCs), including DCs, monocytes, macrophages, neutrophils or myeloid leukemia cells which express LILRB2. Increased antigen presenting activity can lead to T cell activation, cytotoxicity and T cell cytokine production.

In some embodiments, antibodies targeting LILRB2 reprogram immune suppressive myeloid cells to pro-inflammatory in the tumor microenvironment and/or periphery, leading to recruitment and activation of T cells.

In some embodiments, antibodies targeting LILRB2 block binding of one or more ligands implicated in immune-suppressive tumor microenvironment (TME) such as HLA-G, ANGPTL2, CD1c/d, CSPs and SEMA4A.

In some embodiments, antibodies targeting LILRB2 promote activation of primary myeloid and lymphocyte cells.

In some embodiments, antibodies targeting LILRB2 enhance dendritic cell (DC) differentiation, maturation and activation.

In some embodiments, antibodies targeting LILRB2 polarize myeloid cells from solid tumor cancer patients towards a pro-inflammatory phenotype.

In some embodiments, antibodies targeting LILRB2 alleviate the suppressive effect of patient-derived monocytic MDSC (M-MDSC) on autologous T cell proliferation and cytokine release.

In some embodiments, antibodies targeting LILRB2 revert and/or avert the “tumor conditioning” effect of cancer cells on myeloid cells.

In some embodiments, antibodies targeting LILRB2 enhance the effect of pro-inflammatory stimuli, such as anti-CD3 agonist antibody, STING agonist, TLR agonist and anti-PD-1 blocking antibody.

In some embodiments, antibodies targeting LILRB2 inhibit tumor growth in animal models, as single agent or in combination with anti-PD-1, anti-PD-L1, anti-CTLA-4 or other T cell or myeloid checkpoint inhibitors.

Hematologic malignancies include but are not limited to acute lymphocytic/lymphoblastic leukemia (ALL), acute myeloid leukemia (AML), B-cell leukemia, blastic plasmacytoid dendritic cell neoplasm (BPDCN), chronic lymphoblastic leukemia (CLL), chronic lymphocytic leukemia (CLL), chronic myeloid leukemia (CML), chronic myelomonocytic leukemia (CMML), classical Hodgkin lymphoma (CHL), diffuse large B-cell lymphoma (DLBCL), extranodal NK/T-cell lymphoma, hairy cell leukemia, heavy chain disease, HHV8-associated primary effusion lymphoma, lymphoid malignancy, multiple myeloma (MM), myelodysplasia, myelodysplastic syndrome (MDS), non-Hodgkin's lymphoma, plasmablastic lymphoma, pre-B acute lymphocytic leukemia (Pre-B ALL), primary CNS lymphoma, primary mediastinal large B-cell lymphoma, T-cell/histiocyte-rich B-cell lymphoma, myeloproliferative neoplasms, and Waldenstrom's macroglobulinemia.

Chronic viral infection is a disease in which virus is not cleared but remains in specific cells of infected individuals. Chronic viral infection can be caused by a variety of virus es including without limitation Simplex I (HSV-I), Herpes Simplex II (HSV-II), Herpes Virus 3, Herpes Virus 4, Herpes Virus 5, Herpes Virus 6, Parvo Virus B19, Coxsackie A & B, Hepatitis A, Hepatitis B, Hepatitis C, Cytomegalovirus (CMV), and Human Immunodeficiency Virus (HIV).

Autoimmune or inflammatory diseases include, but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, CREST syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma, systemic scleroderma, progressive systemic sclerosis (PSS), systemic sclerosis (SS), Sjogren's syndrome, stiff-man syndrome, systemic lupus erythematosus (SLE), Takayasu arteritis, temporal arteritis/giant cell arteritis, inflammatory bowel disease (IBD), ulcerative colitis, Cohn's disease, intestinal mucosal inflammation, wasting disease associated with colitis, uveitis, vitiligo and Wegener's granulomatosis, Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy, ventilator induced lung injury, viral infections, autoimmune diabetes and the like. Inflammatory disorders include, for example, chronic and acute inflammatory disorders.

The therapeutically effective amount of an antibody or antigen-binding fragment as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the antibody or antigen-binding fragment as provided herein may be administered at a therapeutically effective dosage of about 0.0001 mg/kg to about 100 mg/kg. In certain of these embodiments, the antibody or antigen-binding fragment is administered at a dosage of about 50 mg/kg or less, and in certain of these embodiments the dosage is 10 mg/kg or less, 5 mg/kg or less, 3 mg/kg or less, 1 mg/kg or less, 0.5 mg/kg or less, or 0.1 mg/kg or less. In some embodiments, the antibody or antigen-binding fragment is administered at a dosage of about 4000 mg or less, and in certain of these embodiments, the dosage is about 800 mg or less, about 400 mg or less, about 240 mg or less, about 80 mg or less, 40 mg or less, or 0.8 mg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies and antigen-binding fragments disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the antibodies or antigen-binding fragments disclosed herein may be administered alone or in combination with one or more additional therapeutic means or agents. For example, the antibodies or antigen-binding fragments disclosed herein may be administered in combination with radiation therapy and/or with another therapeutic agent, for example, another immune activator, an anti-angiogenesis agent, a chemotherapeutic agent, or an anti-cancer drug.

In certain of these embodiments, an antibody or antigen-binding fragment as disclosed herein that is administered in combination with one or more additional therapeutic agents may be administered simultaneously with the one or more additional therapeutic agents, and in certain of these embodiments the antibody or antigen-binding fragment and the additional therapeutic agent(s) may be administered as part of the same pharmaceutical composition. However, an antibody or antigen-binding fragment administered “in combination” with another therapeutic agent does not have to be administered simultaneously with or in the same composition as the agent. An antibody or antigen-binding fragment administered prior to or after another agent is considered to be administered “in combination” with that agent as the phrase is used herein, even if the antibody or antigen-binding fragment and second agent are administered via different routes. Where possible, additional therapeutic agents administered in combination with the antibodies or antigen-binding fragments disclosed herein are administered according to the schedule listed in the product information sheet of the additional therapeutic agent, or according to the Prescriber's Digital Reference (available online only at pdr.net) or protocols well known in the art.

In certain embodiments, the agent for combination therapy is an anti-neoplastic composition. As used herein, an “anti-neoplastic composition” refers to a composition useful in treating cancer comprising at least one active therapeutic agent. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents, apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva®), platelet derived growth factor inhibitors (e.g., Gleevec® (Imatinib Mesylate)), a COX-2 inhibitor (e.g., celecoxib), interferons, CTLA4 inhibitors (e.g., anti-CTLA antibody ipilimumab (YERVOY®), or tremelimumab), PD-1 or PD-L1 inhibitors (e.g., OPDIVO® or nivolumab, KEYTRUDA® or pembrolizumab, TECENTRIQ® or atezolizumab, BAVENCIO® or avelumab, IMFINZI® or durvalumab, LIBTAYO® or cemiplimab-rwlc, TYVYT® or sintilimab, tislelizumab (BGB-A317), penpulimab (AK105), camrelizumab, toripalimab, zimberelimab (GLS-010), retifanlimab, sugemalimab, or CS1003), dual-targeting antibodies against CTLA-4 and PD-1 or PD-L1 (e.g., an anti-PD-1/CTLA-4 bi-specific antibody or AK104), TIM3 inhibitors (e.g., anti-TIM3 antibodies), LAG-3 inhibitors (e.g., anti-LAG3 antibodies), cytokines, TLR agonists, STING agonists, antagonists (e.g., neutralizing antibodies) that bind to one or more of the following targets ErbB2, ErbB3, ErbB4, FGFR2b, PDGFR-beta, BlyS, APRIL, BCMA, or VEGF receptor(s), TRAIL/Apo2, an IDH1 inhibitor, an ivosidenib, Tibsovo®, an IDH2 inhibitor, an enasidenib, Idhifa®, a smoothened (SMO) inhibitor, a glasdegib, an arginase inhibitor, an IDO inhibitor, an epacadostat, a BCL-2 inihbitor, a venetoclax, Venclexta®, a platinum complex derivative, oxaliplatin, a kinase inhibitor, a tyrosine kinase inhibitor, a PI3 kinase inhibitor, a BTK inhibitor, an ibrutinib, IMBRUVICA®, an acalabrutinib, CALQUENCE®, a zanubrutinib, an ICOS antibody, a TIGIT antibody, a CD40 antibody, a 4-1BB antibody, a Siglec antibody, an OX40 antibody, a TNFR2 antibody, an antibody to another LILR family member, a CD47 antibody, a SIRPla antibody or fusions protein, an antagonist of E-selectin, an antibody binding to a tumor antigen, an antibody binding to a T cell surface marker, an antibody binding to a myeloid cell or NK cell surface marker, an alkylating agent, a nitrosourea agent, an antimetabolite, an antitumor antibiotic, an alkaloid derived from a plant, a hormone therapy medicine, a hormone antagonist, an aromatase inhibitor, a P-glycoprotein inhibitor and other bioactive and organic chemical agents, etc., an engineered T cell, NK cell or macrophage, a bispecific antibody.

In certain embodiments, the agent for combination therapy is a chemotherapeutic agent. As used herein, a “chemotherapeutic agent” is a chemical compound useful in the treatment of cancer. Examples of chemotherapeutic agents that can be administered in methods herein include, but are not limited to, alkylating agents such as thiotepa and Cytoxan® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, tri etyl enephosphorami de, tri ethiyl enethi ophosphorami de and trimethyl ol omel amine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem Inti. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, Adriamycin® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulini c acid; eniluracil; amsacrine; bestrabucil; bisantrene; edatrax ate ; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), Abraxane® Cremophor-free, albumin-engineered nanoparticle formulation of paclitaxel (American Pharmaceutical Partners, Schaumberg, Illinois), and Taxotere® doxetaxel (Rhone-Poulenc Rorer, Antony, France); chloranbucil; Gemzar® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylomithine (DMFO); retinoids such as retinoic acid; capecitabine; combretastatin; leucovorin (LV); oxabplatin, including the oxaliplatin treatment regimen (FOLFOX); inhibitors of PKC-alpha, Raf, H-Ras, EGFR (e.g., erlotinib (Tarceva))® and VEGF-A that reduce cell proliferation and pharmaceutically acceptable salts, acids or derivatives of any of the above.

Further nonlimiting exemplary chemotherapeutic agents that can be administered in methods herein include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapri stone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

In certain embodiments, the agent for combination therapy is an anti-angiogenesis agent. As used herein, an “anti-angiogenesis agent” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent that can be administered in methods herein can include an antibody or other antagonist to an angiogenic agent, e.g., antibodies to VEGF-A (e.g., bevacizumab (Avastin®)) or to the VEGF-A receptor (e.g., KDR receptor or Flt-1 receptor), anti-PDGFR inhibitors such as Gleevec® (Imatinib Mesylate), small molecules that block VEGF receptor signaling (e.g., PTK787/ZK2284, SU6668, Sutent®/SU1 1248 (sunitinib malate), AMG706, or those described in, e.g., international patent application WO 2004/113304). Anti-angiogenesis agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179; Ferrara & Alitalo (1999) Nature Medicine 5(12): 1359-1364; Tonini et al. (2003) Oncogene 22:6549-6556; and Sato (2003) Int. J. Clin. Oncol. 8:200-206.

In certain embodiments, the agent for combination therapy is a growth inhibitory agent. As used herein, a “growth inhibitory agent” as used herein refers to a compound or composition that inhibits growth of a cell (such as a cell expressing VEGF) either in vitro or in vivo. Thus, the growth inhibitory agent that can be administered in methods herein may be one that significantly reduces the percentage of cells (such as a cell expressing VEGF) in S phase. Examples of growth inhibitory agents include, but are not limited to, agents that block cell cycle progression (at a place other than S phase), such as agents that induce Gl arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest Gl also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W. B. Saunders, Philadelphia, 1995), e.g., p. 13. The taxanes (paclitaxel and docetaxel) are anticancer drugs both derived from the yew tree. Docetaxel (Taxotere®, Rhone-Poulenc Rorer), derived from the European yew, is a semisynthetic analogue of paclitaxel (Taxol®, Bristol-Myers Squibb). Paclitaxel and docetaxel promote the assembly of microtubules from tubulin dimers and stabilize microtubules by preventing depolymerization, which results in the inhibition of mitosis in cells.

The dose of the agent for the combination therapy can be determined by the existence, nature and extent of any adverse side effects that might accompany the administration of a particular agent. Typically, the attending physician will decide the dosage of the agent for the combination therapy with which to treat each individual patient, taking into consideration a variety of factors, such as age, body weight, general health, diet, sex, the agent be administered, route of administration, and the severity of the condition being treated. By way of example and not intending to limit the present disclosure, the dose for the combination therapy can be about 0.0001 to about 1 g/kg body weight of the subject being treated/day, from about 0.0001 to about 0.001 g/kg body weight/day, or about 0.01 mg to about 1 g/kg bodyweight/day. Dosage units may be also expressed in mg/m2, which refer to the quantity in milligrams per square meter of body surface area.

Each therapeutic agent in the combination therapy described herein may be administered simultaneously (e.g., in the same medicament or at the same time), concurrently (i.e., in separate medicaments administered one right after the other in any order or sequentially in any order. Sequential administration may be useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In certain embodiments, the LILRB2 antibody of the present disclosure and the second drug are combined or co-formulated in a single dosage form. In certain embodiments, the LILRB2 antibody of the present disclosure and the second drug are administered separately. Although the simultaneous administration of the LILRB2 antibody of the present disclosure and the second drug may be maintained throughout a period of treatment, anti-cancer activity may also be achieved by subsequent administration of one compound in isolation (for example, the LILRB2 antibody following initial combination treatment, or alternatively, the second drug following initial combination treatment). In some embodiments, the LILRB2 antibody is administered before administration of the second drug, while in other embodiments, the LILRB2 antibody is administered after administration of the second drug. In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

The combination therapy of the invention may be used prior to or following surgery to remove a tumor and may be used prior to, during or after radiation therapy. The combination therapy of the invention may be used to treat a tumor that is large enough to be found by palpation or by imaging techniques well known in the art, such as Mill, ultrasound, or CAT scan. In some embodiments, the combination therapy of the invention is used to treat an advanced stage tumor having dimensions of at least about 200 mm³, 300 mm³, 400 mm³, 500 mm³, 750 mm³, or up to 1000 mm³.

The present disclosure further provides methods of using the anti-LILRB2 antibodies or antigen-binding fragments thereof to detect presence or amount of LILRB2 in a sample, comprising contacting the sample with the antibody or antigen-binding fragment thereof, and determining the presence or the amount of LILRB2 in the sample. The method of detecting LILRB2 using an anti-LILRB2 antibody includes, without limitation, ELISA, RIA, Western-blot, flow cytometry and immunohistochemistry.

In some embodiments, the present disclosure provides methods of diagnosing a LILRB2 related disease or condition in a subject, comprising: a) contacting a sample obtained from the subject with the antibody or antigen-binding fragment thereof provided herein; b) determining presence or amount of LILRB2 in the sample; and c) correlating the existence of the LILRB2 to the LILRB2 related disease or condition in the subject.

In some embodiments, the present disclosure provides kits comprising the antibody or antigen-binding fragment thereof provided herein, optionally conjugated with a detectable moiety. The kits may be useful in detection of LILRB2 or diagnosis of LILRB2 related disease.

In some embodiments, the present disclosure also provides use of the antibody or antigen-binding fragment thereof provided herein in the manufacture of a medicament for treating a LILRB2 related disease or condition in a subject, in the manufacture of a diagnostic reagent for diagnosing a LILRB2 related disease or condition.

### V. Chimeric Antigen Receptors

The present disclosure in another aspect provides a chimeric antigen receptor (CAR) protein that binds LILRB2 (anti-LILRB2 CAR protein). In certain embodiments, the CAR protein comprises an antigen recognition region, i.e., an antibody or antigen-binding fragment that recognizes LILRB2 as described herein, and other membrane and intracellular components. In some embodiments, the anti-LILRB2 CAR protein comprises a LILRB2 antigen recognition region, a transmembrane domain and an intracellular co-stimulatory signal domain. In certain embodiments, the single chain anti-LILRB2 CAR protein also comprises a leader peptide, a spacer region and an intracellular T cell signaling domain.

In certain embodiments, the antigen recognition region comprises multiple polypeptide chains.

In some embodiments, the CAR protein comprises a first polypeptide including an antibody heavy chain variable domain and a polypeptide including an antibody light chain variable domain, wherein the first or the second polypeptide further includes a transmembrane domain, and wherein the antibody heavy chain variable domain and the antibody light chain variable domain together form an antigen recognition region.

In some embodiments, the CAR protein comprises a first polypeptide including an antibody heavy chain variable domain and a second polypeptide including an antibody light chain variable domain and an antibody light chain constant domain, wherein the first polypeptide further includes a transmembrane domain, and wherein the antibody heavy chain variable domain, the antibody light chain variable domain and the antibody light chain constant domain together form an antigen recognition region. In some embodiments, the first portion further includes an intracellular co-stimulatory signaling domain and a CD3ζ intracellular T cell signaling domain.

In some embodiments, the CAR protein comprises a first polypeptide including an antibody heavy chain variable domain and an antibody heavy chain constant domain, and a second polypeptide including an antibody light chain variable domain, wherein the first polypeptide further includes a transmembrane domain, and wherein the antibody heavy chain variable domain, the antibody heavy chain constant domain, and the antibody light chain variable domain together form an antigen recognition region. In some embodiments, the first portion further includes an intracellular co-stimulatory signaling domain and a CD3ζ intracellular T cell signaling domain.

In some embodiments, the CAR protein comprises a first polypeptide including an antibody heavy chain variable domain and a second polypeptide including an antibody light chain variable domain, wherein the second polypeptide further includes a transmembrane domain, and wherein the antibody heavy chain variable domain, the antibody light chain variable domain and the antibody light chain constant domain together form an antigen recognition region. In some embodiments, the second portion further includes an intracellular co-stimulatory signaling domain and a CD3ζ intracellular T cell signaling domain.

In some embodiments, the CAR protein comprises a first polypeptide including an antibody heavy chain variable domain and an antibody heavy chain constant domain, and a second polypeptide including an antibody light chain variable domain, wherein the second polypeptide further includes a transmembrane domain, and wherein the antibody heavy chain variable domain, the antibody heavy chain constant domain, and the antibody light chain variable domain together form an antigen recognition region. In some embodiments, the second portion further includes an intracellular co-stimulatory signaling domain and a CD3ζ intracellular T cell signaling domain.

In certain embodiments, the CAR protein is a single chain polypeptide that comprises an anti-LILRB2 scFv as described herein, i.e., an anti-LILRB2 heavy chain variable domain and an anti-LILRB2 light chain variable domain, which are linked by a linker domain. In one embodiment, the CAR protein includes from the N-terminus to the C-terminus: a leader peptide, an anti-LILRB2 heavy chain variable domain, a linker domain, an anti-LILRB2 light chain variable domain, a hinge region, a transmembrane domain, an intracellular co-stimulatory signal domain. In one embodiment, the CAR protein includes from the N-terminus to the C-terminus: a leader peptide, an anti-LILRB2 light chain variable domain, a linker domain, an anti-LILRB2 heavy chain variable domain, a hinge region, a transmembrane domain, an intracellular co-stimulatory signal domain. In some embodiments, the CAR protein further includes a CD3ζ intracellular T cell signaling domain.

In certain embodiment, the linker domain generally is comprised of helix- and turn-promoting amino acid residues such as alanine, serine and glycine. However, other residues can function as well. In some embodiment, the linker domain is inserted between the VH and VL of the scFv. In some embodiments, the linker domain is between the transmembrane domain and the intracellular co-stimulatory signaling domain. In some embodiments, the linker domain is between the intracellular T cell signaling domain and the intracellular co-stimulatory signaling domain. In some embodiments, the linker domain comprises the sequence GGGGSGGGGSGGGGS (SEQ ID NO: 128).

In some embodiments, the transmembrane domain is a CD8a transmembrane domain which has at least 90%, 95%, 96%, 97%, 98%, 99% or 100% amino acid sequence identity compared to a naturally occurring CD8a transmembrane domain polypeptide (SEQ ID NO: 129). In some embodiments, the CD8a transmembrane domain is encoded by the nucleic acid sequence of SEQ ID NO: 130.

In some embodiments, the transmembrane domain is a CD28 transmembrane domain which has at least 90%, 95%, 96%, 97%, 98%, 99% or 100% amino acid sequence identity compared to a naturally occurring CD28 transmembrane domain polypeptide (SEQ ID NO: 131). In some embodiments, the CD28 transmembrane domain is encoded by the nucleic acid sequence of SEQ ID NO: 132.

The intracellular co-stimulatory signaling domain includes amino acid sequences capable of providing co-stimulatory signaling in response to binding of an antigen to the CAR. In some embodiments, the signaling of the co-stimulatory signaling domain results in the production of cytokines and proliferation of the T cell or NK cell expressing the same. In some embodiments, the intracellular co-stimulatory signaling domain is a CD28 intracellular co-stimulatory signaling domain, a 4-1BB intracellular co-stimulatory signaling domain, an ICOS intracellular co-stimulatory signaling domain, an OX-40 intracellular co-stimulatory signaling domain or any combination thereof. In some embodiments, the CD28 co-stimulating domain has the polypeptide sequence of SEQ ID NO: 133. In some embodiments, the CD28 intracellular co-stimulatory signaling domain is encoded by the nucleic acid sequence of SEQ ID NO: 134. In some embodiments, the 4-1BB intracellular co-stimulatory signaling domain has the polypeptide sequence of SEQ ID NO: 135. In some embodiments, the 4-1BB intracellular co-stimulatory signaling domain is encoded by the nucleic acid sequence of SEQ ID NO: 136.

A “hinge region” as provided herein is a polypeptide connecting the antigen-binding region with the transmembrane domain. In some embodiments, the hinge region connects a heavy chain variable region with the transmembrane domain. In some embodiments, the hinge region connects a heavy chain constant region with the transmembrane domain. In some embodiments, the hinge region connects a light chain variable region with the transmembrane domain. In some embodiments, the hinge region connects a light chain constant region with the transmembrane domain. In some embodiments, the binding affinity of the antigen-binding region to an antigen is increased compared to the absence of the hinge region. In some embodiments, the steric hindrance between an antigen-binding region and an antigen is decreased in the presence of the hinge region. In some embodiments, the hinge region is a CD8a hinge region. In some embodiments, the hinge region is a CD28 hinge region.

In some embodiments, the intracellular T cell signaling domain includes the signaling domain of the zeta (C) chain of the human CD3 complex, i.e., a CD3ζ intracellular T cell signaling domain. In some embodiments, the intracellular T cell signaling domain is the protein CD3zIso1 with the amino acid sequence of SEQ ID No: 137. In some embodiments, the intracellular T cell signaling domain is the protein CD3zIso3 with the amino acid sequence of SEQ ID No: 138, encoded by the nucleic acid sequence of SEQ ID NO: 139.

In one example, the CAR protein is a single chain polypeptide that includes from the N-terminus to the C-terminus: a CD8a leader peptide, an anti-LILRB2 scFv, a CD8a hinge region, a CD8a transmembrane domain (or a CD28 transmembrane domain), a 4-1BB intracellular co-stimulatory signaling domain (or a CD28 intracellular co-stimulatory signaling domain, or a CD28 intracellular co-stimulatory signaling domain followed by a 4-1BB intracellular co-stimulatory signaling domain) and a CD3ζ intracellular T cell signaling domain in one of two isoforms (CD3zIso1 or CD3zIso3).

In certain embodiments, the anti-LILRB2 CAR protein provided herein demonstrates a high binding affinity to LILRB2. In certain embodiments, the CAR protein provided herein has a binding affinity to LILRB2 (EC₅₀ as measured by ELISA) of less than 1 nM, 0.9 nM, 0.8 nM, 0.7 nM, 0.6 nM, 0.5 nM, 0.4 nM, 0.3 nM, 0.2 nM, 0.1 nM, 0.09 nM, 0.08 nM, 0.07 nM, 0.06 nM or 0.05 nM. For the purposes of this application, ELISA ECso values may be determined as follows. Recombinant LILRB2 ECD-6× His tagged protein is coated onto a high binding 96-well clear plate (Corning-Costar, Fisher Scientific) at 1 μg/ml concentration (100 μl/well) at 4° C. for 14 to 16 hours. The coated plates are washed with PBS, pH 7.4, briefly and blocked with 200 μl/well of 5% non-fat milk in PBS for 2 hours at 37° C. Serial dilutions of the testing monoclonal antibodies (IgGs or scFvs fragments), starting from 10 μg/ml and 3-fold titration down for 12 steps, are added to the 96-well plate for binding by incubating 45 minutes at 37° C. with a cover on the assay plate. Then the plates are washed with PBS containing Tween 20 (0.05% concentration) for 3 times and PBS one time. Secondary antibody of anti-human or anti-rabbit, or other species IgG specific antibodies with HRP conjugate (Jackson ImmunoResearch) is added for incubation at room temperature for 1 hour per manufacturer's suggested dilution. Detection is conducted by adding HRP substrate, tetramethylbenzidine (TMB, ThermoFisher) for 10 minutes, and stopped by adding 50 μl/well of 2N H₂SO₄. The plates are read for absorbance at 450 nm using a plate reader (SpectraMax M4, Molecular Devices). Data are collected and graphed using a 4-parameter fitting curve with GrapPad Prism 7 software for EC₅₀ calculation.

In another aspect, the present disclosure provides a polynucleotide molecule encoding a CAR protein described herein. In some embodiments, the polynucleotide molecule further comprises a promoter active in eukaryotic cells. In some embodiments, the promoter is the JeT promoter. The JeT promoter is a recombinant promoter with transcriptional activity comparable to a number of strong mammalian promoters. The JeT promoter consists of five key elements: (1) a TATA box; (2) a transcription initiation site (Inr); (3) a CAT consensus sequence in conjunction with (4) a CArG element and finally, (5) four Spl transcription binding sites (GGGCGG) arranged in two tandems (US 2002/0098547 A1). In some embodiments, the polynucleotide molecule is an expression vector. In some embodiment, the vector is generated based on pLVX-EFlalpha-IRES-ZsGreen from Clontech, or pSIN-EFlalpha-IRES-Puromycin or pSIN-EFlalpha. In one example, the polynucleotide molecule of the present disclosure comprises the following elements sequentially: (1) JeT promoter; (2) sequence encoding a CD8-alpha leader; (3) sequence encoding a heavy chain variable region; (4) sequence encoding a linker; (5) sequence encoding a light chain variable region; (6) sequence encoding a CD8 hinge and TM domain; (7) sequence encoding a 4-1BB co-stimulatory domain; (8) sequence encoding a CD3-zeta activation domain. In one example, the elements described above are flanked by 5′ and 3′ homologous arms that facilitate the insertion of the polynucleotide molecule to a target locus, e.g., T cell receptor alpha constant (TRAC) locus.

### VI. Engineered Cells Expressing Anti-LILRB2 CAR Protein

In another aspect, the present disclosure provides engineered immune cells which express a CAR protein described herein. The immune cells may be T cells (e.g., regulatory T cells, CD4⁺ T cells, CD8⁺ T cells, or gamma-delta T cells), Natural Killer (NK) cells, invariant NK cells, NKT cells, or macrophages. Also provided herein are methods of producing and engineering the immune cells as well as methods of using and administering the cells for adoptive cell therapy, in which case the cells may be autologous or allogeneic. Thus, the engineered immune cells may be used as immunotherapy, such as to target LILRB2⁺ cancer cells.

Expressing the CAR protein allows the engineered immune cells to bind to a target cell, such as a cancer cell, by recognizing an antigen present on the target cell. Upon binding to the target cell, the engineered immune cell becomes activated, then proceed to proliferate and become cytotoxic, eventually destroys the target cell. CAR-T cell immunotherapy has demonstrated success in clinical trials and been approved by U.S. FDA to treat refractory B-cell acute lymphoblastic leukemia and B-cell non-Hodgkin lymphoma (Hartmann J et al., EMBO Mol Med (2017) 9:1183-97). CAR NK cells and CAR macrophages have been developed recently as immunotherapy options in addition to CAR-T cells (Kloess S et al., Transfusion Medicine and Hemotherapy (2019) 46:4-13; Klichinsky M et al., AACR Annual Meeting 2017, Abstract 4575). Therefore, in certain embodiments of the present disclosure, the immune cells that express the CAR protein described herein are T cells, NK cells or macrophages.

The immune cells may be isolated from subjects, particularly human subjects. The immune cells can be obtained from a subject of interest, such as a subject suspected of having a particular disease or condition, a subject suspected of having a predisposition to a particular disease or condition, a subject who is undergoing therapy for a particular disease or condition, a subject who is a healthy volunteer or healthy donor, or from blood bank. Immune cells can be collected from any location in which they reside in the subject including, but not limited to, blood, cord blood, spleen, thymus, lymph nodes, and bone marrow. The isolated immune cells may be used directly, or they can be stored for a period of time, such as by freezing.

The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. In particular embodiments, the immune cells are isolated from blood, such as peripheral blood or cord blood. In some aspects, immune cells isolated from cord blood have enhanced immunomodulation capacity, such as measured by CD4- or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor, preferably a histocompatibility matched donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the population of immune cells is obtained from a donor distinct from the subject, the donor is preferably allogeneic, provided the cells obtained are subject-compatible in that they can be introduced into the subject. Allogeneic donor cells may or may not be human-leukocyte-antigen (HLA)-compatible. To be rendered subject-compatible, allogeneic cells can be treated or genetically manipulated, e.g., deleting T cell receptors or inhibiting the T cell signaling pathway, to minimize graft vs host disease (see Kim and Cho, Recent Advances in Allogeneic CAR-T Cells, Biomolecules (2020) 10:263).

The immune cells can be genetically engineered to express the CARs using suitable methods of modification are known in the art. See, for instance, Sambrook and Ausubel, CURRENT PROTOCOLS IN MOLECULAR BIOLOGY, Greene Publishing Associates and John Wiley & Sons, NY, 1994. In some embodiments, the immune cells comprise one or more nucleic acids introduced via genetic engineering that encode one or more CAR proteins. In certain embodiments, the nucleic acids encoding the CAR proteins are inserted in the genome of the immune cells using gene editing methods, e.g. CRISPR/Cas technology. In one example, the nucleic acids encoding the CAR proteins are inserted at the T cell receptor alpha constant (TRAC) locus (see, e.g., Eyquem Jet al., Nature (2017) 543:113-117).

Also provided are methods for immunotherapy comprising administering an effective amount of the immune cells of the present disclosure. In some embodiments, a medical disease or disorder is treated by transfer of a population of immune cells described herein that elicits an immune response. In certain embodiments, the medical disease or disorder is a cancer. In certain embodiments, the medical disease or disorder is an autoimmune or inflammatory disease.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLE 1

This example illustrates the design of the anti-LILRB2 antibody variants.

The inventors previously identified an anti-LILRB2 antibody named B2-19 that has a high binding affinity to LILRB2 (see PCT Patent Application No. PCT/US2021/015362, the disclosure of which is incorporated herein in its entirety). Analyzing the variable domain sequences of the parent phage display-derived B2-19 antibody led to the identification of isomerization (DG, DD, DD, DS), deamidation (NN, NG), and oxidation (W) motifs which have been reported to be liabilities in other clinical-stage antibodies (Lu et al mAbs 2019, 11:1, 45-57 and Yang et al mAbs 2017, 9:4, 646-653). Analysis of the variable domains of B2-19 using IgBLAST (Ye et al Nucleic Acids Res 2013 W34:W40) also identified mutations in the framework regions which could present an immunogenicity risk. Accordingly, point mutations of B2-19 were designed to eliminate liabilities in the CDRs: isomerization motifs (B2-19-1 through B2-19-6), deamidation motifs (B2-19-7 through B2-19-8), oxidation sites (B2-19-9 and B2-19-10). Framework residues in the VH and VL variable domains were germlined to minimize unnecessary immunogenicity risk (B2-19-11 or germlined/germ). Antibody variants were produced by cloning into a pcDNA3.4 vector, expressing in Mammalian Expi293F system, and purifying with a RoboColumn Eshmuno A 0.6 mL. Binding affinities were measured by BLI using a Gator system with an anti-human IgG Fc (HFC) probe loading the antibody at 5 μg/mL and titrating recombinant LILRB2 ECD-6× His tagged protein (R&D systems cat 8429-T4). Data was analyzed with global fit. B2-19-12 was designed by combining mutations which reduced immunogenicity risk and eliminated all isomerization motifs without significant loss in binding affinity.

Additional variants were designed to further optimize B2-19. First, variants 14-18 layered additional mutations on variant 12 to remove deamidation motifs. Variants 20-32 and 41-63 were designed to reduce surface-exposed hydrophobic and Arg and Lys residues which have been correlated with higher polyspecificity and poor pharmacokinetics (Sharma et al PNAS 2014, 111, 52, 18601-18606 and Shehata et al Cell Reports 2019 28, 3300-3308). To potentially improve thermal stability, variants 34 and 35 were engineered by grafting the B2-19 lambda light-chain CDRs onto the human kappa 1-39 and 3-20 light-chain frameworks using methods described previously (Lehmann et al mAbs 7:6 1058-1071). Similarly, variants 38-40 were designed by grafting CDRs onto VH3-23 and VL2-23 frameworks which have been reported to have good biophysical properties in clinical stage antibodies (Jain et al PNAS 2017, 114, 5, 944-949). Variants 36 and 37 were designed to improve the Fv charge symmetry parameter (FvCSP) to potentially lower viscosity in high concentration formulations (Sharma et al PNAS 2014, 111, 52, 18601-18606). All these antibody variants were cloned into a custom antibody vector based on one described in the literature (Tiller et al J Immunol Methods 2008, 329, 112-124) and expressed using a Expi293 expression system (-6 mL culture, ThermoFisher cat A14635). As shown in Table 1, off-rates were measured from the supernatant by Bio-Layer Interferometry (BLI) using a Gator system with an HFC probe loading the antibody variants and measuring binding to recombinant LILRB2 ECD-6× His tagged protein (R&D systems cat 8429-T4).

TABLE 1
Off-rates measured by BLI were used to select variants
with similar kinetics to the parent B2-19 antibody.

| Antibody Name | Off-rate (koff 1/s) |
|---|---|
| B2-19 | 2.2E−04 |
| B2-19-1 | 7.4E−04 |
| B2-19-2 | 8.4E−04 |
| B2-19-3 | 5.5E−04 |
| B2-19-4 | 1.7E−03 |
| B2-19-5 | 2.4E−04 |
| B2-19-6 | 3.6E−04 |
| B2-19-7 | 3.0E−04 |
| B2-19-8 | 1.9E−04 |
| B2-19-9 | 9.0E−03 |
| B2-19-10 | 1.3E−03 |
| B2-19-11 | 2.2E−04 |
| B2-19-12 | 4.2E−04 |
| B2-19-14 | 5.5E−04 |
| B2-19-15 | 1.5E−03 |
| B2-19-16 | 3.0E−04 |
| B2-19-17 | 1.7E−03 |
| B2-19-18 | 1.7E−03 |
| B2-19-20 | 1.1E−02 |
| B2-19-21 | NO BINDING |
| B2-19-22 | 6.8E−04 |
| B2-19-23 | 6.9E−03 |
| B2-19-24 | NO BINDING |
| B2-19-25 | 8.9E−03 |
| B2-19-26 | 9.6E−03 |
| B2-19-28 | 2.6E−03 |
| B2-19-30 | 5.4E−03 |
| B2-19-31 | NO BINDING |
| B2-19-32 | 6.2E−03 |
| B2-19-34 | 1.4E−02 |
| B2-19-35 | 4.0E−03 |
| B2-19-36 | 5.0E−04 |
| B2-19-37 | 3.3E−02 |
| B2-19-38 | 1.3E−03 |
| B2-19-39 | 3.5E−03 |
| B2-19-40 | 1.8E−02 |
| B2-19-41 | 7.0E−04 |
| B2-19-42 | 6.4E−04 |
| B2-19-43 | 1.5E−03 |
| B2-19-44 | 4.8E−03 |
| B2-19-45 | 1.2E−03 |
| B2-19-46 | 1.0E−03 |
| B2-19-48 | 1.6E−02 |
| B2-19-49 | 1.6E−02 |
| B2-19-50 | 4.7E−03 |
| B2-19-51 | 7.0E−03 |
| B2-19-52 | 6.7E−03 |
| B2-19-53 | 6.3E−04 |
| B2-19-54 | 1.1E−03 |
| B2-19-55 | 4.0E−04 |
| B2-19-56 | 1.7E−02 |
| B2-19-57 | 1.1E−02 |
| B2-19-58 | 8.4E−03 |
| B2-19-59 | NO BINDING |
| B2-19-60 | 2.6E−02 |
| B2-19-61 | 3.1E−03 |
| B2-19-62 | NO BINDING |
| B2-19-63 | 2.3E−02 |

### EXAMPLE 2

This example illustrates binding affinity, specificity and biological properties of B2-19 antibody variants.

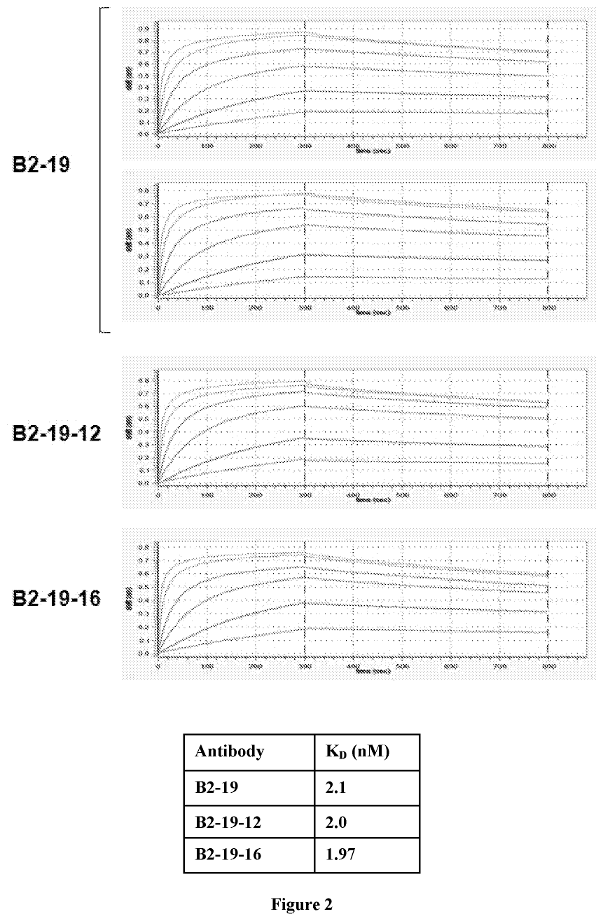

The binding affinities of B2-19 and select variants to recombinant LILRB2 ECD-6× His tagged protein were measured by BLI. As shown in FIG. 2, the B2-19 antibody variants have the same binding affinity to LILRB2 as the parent B2-19 antibody. The measured affinities were all similar, within experimental error, with a K_{D} of approximately 2.0 nM.

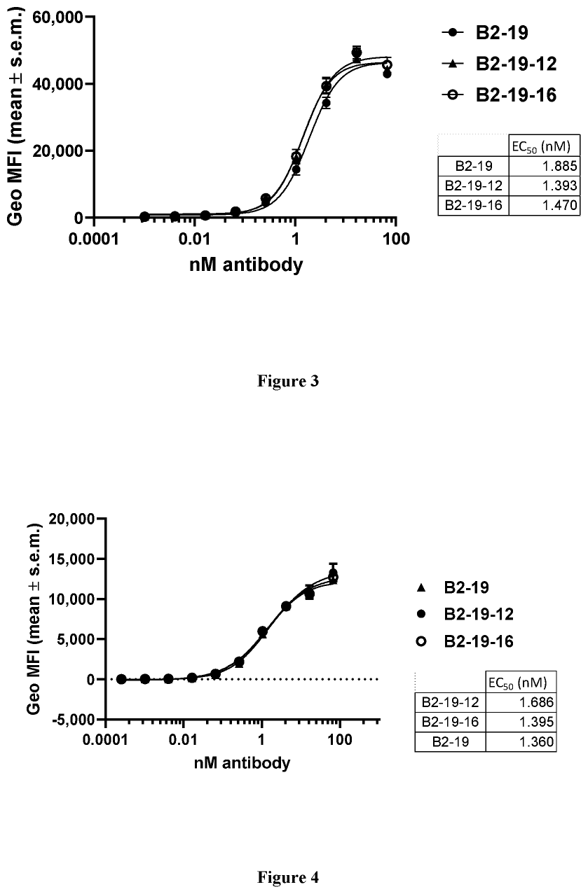

Binding potency of the B2-19 antibody variants on HEK293 cells stably expressing LILRB2 was analyzed by flow cytometric. Fifty thousand cells were incubated for 30 min. at 4° C. with a 4-fold dilution series (10-0.00015 μg/mL) of test anti-LILRB2 antibodies in a final volume of 100 pL. After washing, bound antibodies were detected using a goat anti-human Fc-specific secondary antibody conjugated to Alexa Fluor 647. Results were analyzed using FlowJo software and dose-response curves plotted in GraphPad Prism. As shown in FIG. 3, the B2-19 antibody and its variants have comparable binding potency to LILRB2 stably expressed on HEK293 cells. Data shown is averaged geometric 1VIFI from samples tested in duplicate plates and analyzed in a flow cytometer (BD FACS Celesta).

To measure the binding potency of the B2-19 antibody variants on monocytes, 50,000 CD14⁺CD16⁻ monocytes were isolated from healthy donors' PBMC, pre-incubated with 400 μg/mL human IgG from human serum for 10 min. at 4° C. to block Fc gamma receptors and then immediately incubated for 30 min. at 4° C. with a 4-fold dilution series (10-0.000038 μg/mL) of test anti-LILRB2 antibodies directly conjugated to Alexa Fluor 647 in a final volume of 100 μL. Data shown is averaged geometric MFI from duplicate samples acquired in a flow cytometer (BD FACS Celesta) from one donor. Results were analyzed using FlowJo software and dose-response curves plotted in GraphPad Prism. As shown in FIG. 4, the B2-19 antibody and its variants have comparable binding potency to endogenous LILRB2 on primary CD14⁺CD16⁻ monocytes.

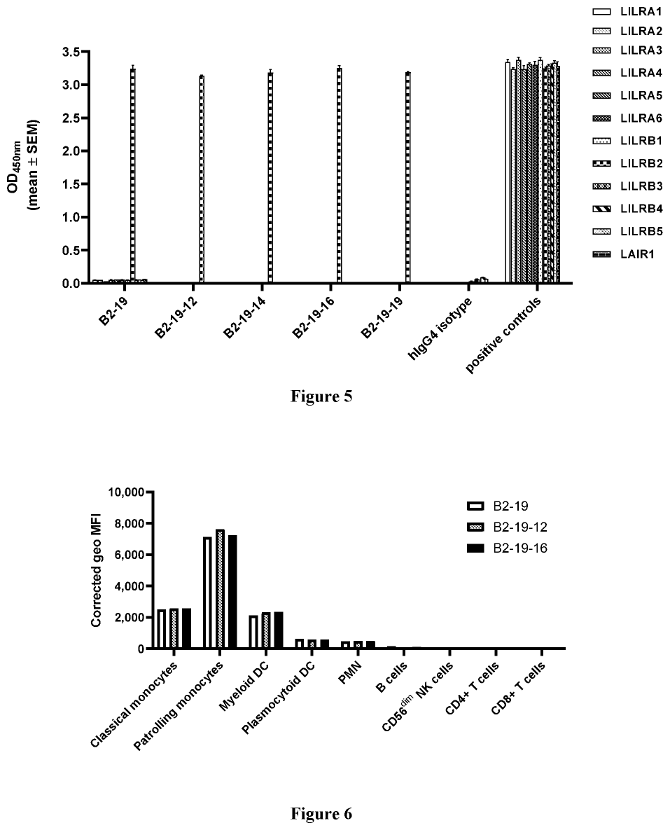

The binding specificity of the B2-19 antibody variants to LILRB2 was measured by ELISA. Recombinant ECD of LILRs/LAIR1 merged at C-terminus with 6× His-tag proteins were coated on ELISA plates at 5 μg/mL and incubated with antibodies at a concentration of 10 nM (10-fold above concentration resulting in binding saturation of B2-19 antibody to LILRB2, as determined in pilot experiments) and incubated for 2 hours at room temperature. Bound antibodies were detected by HRP-conjugated goat anti-human Fc-specific secondary antibody and TMB substrate. Optical density at 450 nm was measured in a SpectraMax M5 Spectrophotometer (Molecular Devices) and data analyzed with SoftMax Pro. As shown in FIG. 5, the B2-19 antibody and its variants bind specifically to LILRB2.

To measure the binding specificity of the B2-19 antibody variants to myeloid cells, the reactivity of the antibodies on leukocytes from whole blood harvested from healthy donors was characterized by flow cytometry. B2-19 antibody and its variants were directly conjugated with Alexa Fluor 647. One hundred microliters of whole blood were incubated with antibodies for cell surface markers and anti-LILRB2 antibodies, following protocols available in the literature (Hensley et al., J Vis Exp 2012; 67: 4302). Samples were analyzed in a flow cytometer (BD FACS Celesta) and results analyzed using FlowJo software. As shown in FIG. 6, the B2-19 antibody and its variants bind specifically to myeloid cells in whole blood. Data shown is corrected geometric MFI of sample, i.e., geometric MFI of anti-LILRB2 stained samples subtracted by geometric MFI of samples in which LILRB2 antibodies were omitted (FMO control). Representative data from one donor is shown (N=3 donors).

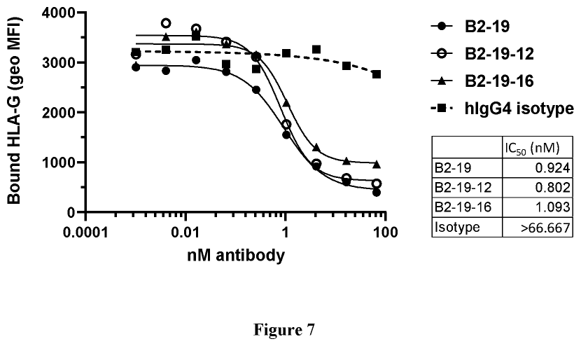

To measure the activity of the B2-19 antibody variants in blocking LILRB2 binding to HLA-G, 50,000 HEK293 cells stably expressing LILRB2 were incubated with His-tagged soluble HLA-G at 10 μg/mL (corresponding to ˜EC₈₀ of binding, as determined in pilot experiments) in the presence of a 4-fold dilution series (10-0.00015 μg/mL) of anti-LILRB2 antibodies. After washing, bound HLA-G was detected using an anti-His antibody directly conjugated to allophycocyanin (APC). All incubations were performed for 30 min. at 4° C. Samples were analyzed in a flow cytometer (BD FACS Celesta). Results were analyzed using FlowJo software and dose-response curves plotted in GraphPad Prism. As shown in FIG. 7, the B2-19 antibody and its variants have comparable blocking activity of HLA-G binding.

To measure the pro-inflammatory effect of the B2-19 antibody variants, PBMC were isolated from healthy donors using Ficoll-Paque Plus (GE Healthcare) and incubated in 96-well round bottom plates for 3 days in the presence of 10 ng/mL anti-CD3 mAb (HIT3a) in the presence of anti-LILRB2 antibodies or isotype control. Cytokine concentration levels were measured in the culture media supernatant at the end of the 3-day incubation using a Human Cytokine Premixed Magnetic Luminex Performance Assay (R&D Systems). As shown in FIGS. 8A and 8B, the B2-19 and B2-19-16 antibodies have comparable pro-inflammatory effect on PBMC isolated from healthy donors and stimulated with a sub-optimal anti-CD3 mAb concentration.

### EXAMPLE 3

This example illustrates the reduced polyspecificity of the anti-LILRB2 antibody variants.

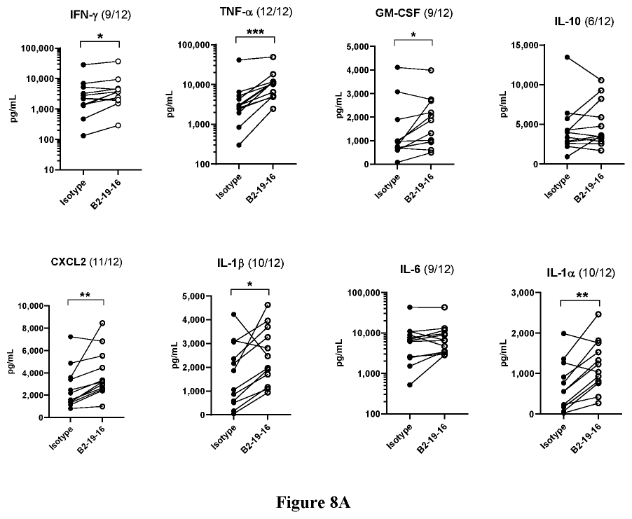

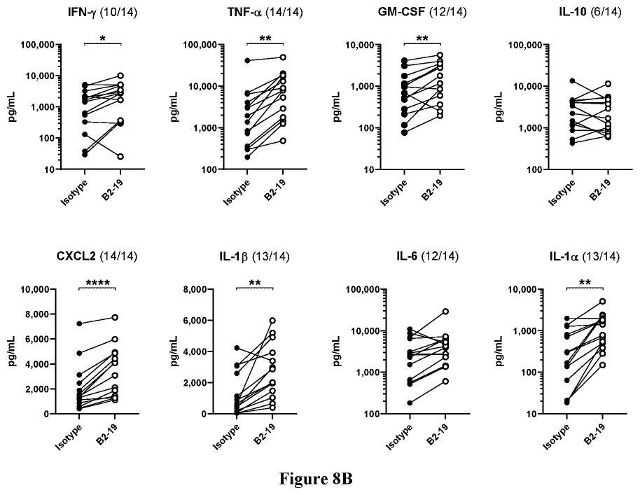

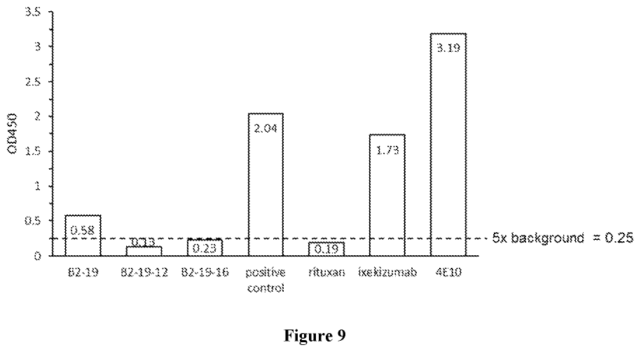

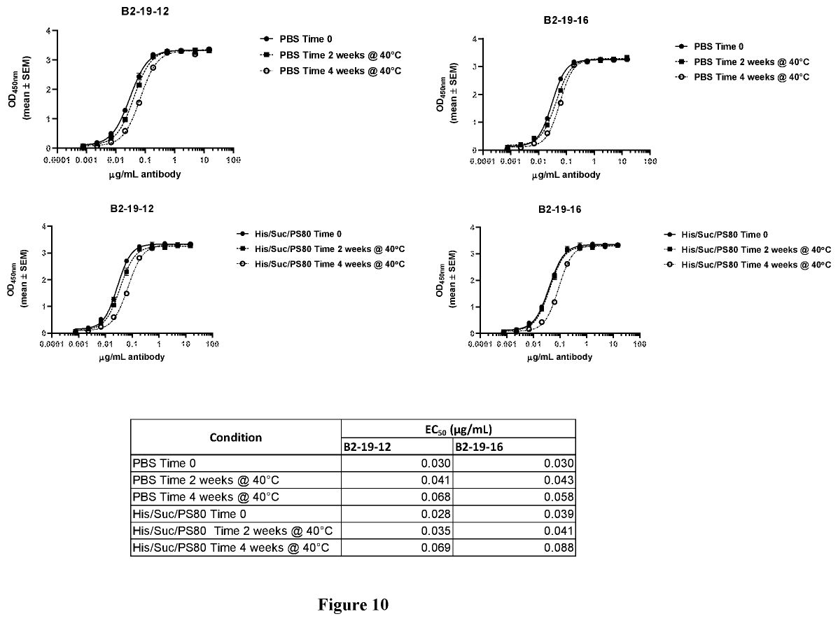

High BVP polyspecificity ELISA scores of antibodies have shown a correlation to rapid clearance in cynomolgus monkeys and humans (Hotzel et al mAb 2012, 4, 6, 753-760) and shorter half-life in humans (Shehata et al Cell Reports 2019 28, 3300-3308). Thus, a BVP ELISA was performed to assess polyspecificity of B2-19 and select variants to determine which had a lower risk of exhibiting poor pharmacokinetics (PK) in humans. Baculovirus particles were produced by LakePharma. Briefly, 2L of Sf9 cells were cultured with baculovirus, isolated, and resuspended in PBS pH 7.4 and stored at −80° C. The total protein of the BVP prep was measured using a Bradford as to be 2.3 mg/mL and the titer was 5.71×10¹² pfu/mL. The BVP ELISA was performed as described previously (Hotzel et al mAb 2012, 4, 6, 753-760). Briefly, a MaxiSorp plate was coated with 0.5% BVP in carbonate buffer pH 9.6 at 4° C. overnight, washed once with 300 μL/well PBS and blocked with for one hour with 200 μL/well PBS+0.5% BSA. After washing the blocked plate 3 times with 300 μL/well PBS, 150 μg/mL antibody in PBS+0.5% BSA was incubated for 1 hour, followed by 6 washes with 300 μL/well PBS. Bound antibody was detected for 1 hour after adding 100 μL/well of a goat anti-human Fc-HRP conjugate diluted 1/20,000 or 1/40,000 in PBS+0.5% BSA. The plate was developed with TMB after 3 washes with 300 μL/well PBS, and stopped with hydrochloric acid after 10 min. BVP scores were calculated by dividing the OD 450 nm of the sample by the background signal for the secondary antibody alone on BVP. At the 1/20,000 secondary dilution, the BVP score of the wt B2-19 is above the 5-fold cut-off indicating it has a risk for poor PK in humans and non-human primates. As shown in FIG. 9 and Table 2, B2-19-12 and B2-19-16 both have BVP scores below the 5-fold cut-off indicating they have lower risks of having poor PK. A commercially available positive control (MEDNA cat H1308) and other therapeutic antibodies (e.g., Rituxan, lxekizumab, 4E10) were used in the assays as references to correlate with literature values (Jain et al PNAS 2017, 114, 5, 944-949 and Shehata et al Cell Reports 2019 28, 3300-3308).

TABLE 2
BVP Scores for B2-19 and its variants compared to controls

| Antibody | BVP score |
|---|---|
| B2-19 | 11.6 |
| B2-19-12 | 2.5 |
| B2-19-16 | 4.7 |
| Positive control (MEDNA cat# H1308) | 41.0 |
| Rituxuan | 3.9 |
| Ixekizumab | 34.7 |
| 4E10 | 64.1 |

### EXAMPLE 4

This example illustrates the thermal stability of anti-LILRB2 antibody variants.

To assess the thermal stability of B2-19-12 and B2-19-16, the antibodies were incubated for 4 weeks at 40° C. in PBS pH 7.5 or formulation buffer (FB, 20 mM histidine-HCl, 7% sucrose, 0.02% w/v PS80, pH 5.5) at ˜2 mg/mL. Initial samples (TO) and aliquots collected after 2 (2W) and 4 (4W) weeks, were analyzed by size-exclusion chromatography (SEC) for aggregation and fragmentation. As shown in Table 3, in PBS and FB an increase in the percent high molecular weight (%HMW) was observed for both molecules but remained under 6%. There was also a slight increase in percent low molecular weight (%LMW), but this stayed less than 0.6%. The binding activities of the antibodies were also assessed by ELISA in PBS and FB before and after incubation at 40° C. As shown in Figure slight decrease in binding EC₅₀ (˜2-3-fold) was observed after the 4-week incubations. Overall, these results suggest that both molecules do not aggregate, fragment, or lose binding activity significantly over time under thermal stress.

TABLE 3
B2-19 antibody variants demonstrate low tendency
to aggregate or fragment upon thermal stress.

| | SEC-HPLC (Main peak %/HMW %/LMW %) | | | | | |
|---|---|---|---|---|---|---|
| | Buffer: PBS | | | Buffer: FB | | |
| | | 40° C. | | | 40° C. | |
| Molecule | T0 | 2 W | 4 W | T0 | 2 W | 4 W |
| B2-19-12 | 96.0/4.0/0.0 | 94.1/5.8/0.2 | 92.5/7.0/0.6 | 97.7/2.3/0.0 | 97.0/2.8/0.2 | 95.9/3.7/0.4 |
| | | (−1.9) | (−3.5) | | (−0.7) | (−1.8) |
| B2-19-16 | 96.8/3.2/0.0 | 93.4/6.1/0.4 | 91.2/8.2/0.6 | 97.9/2.1/0.0 | 97.1/2.8/0.1 | 95.3/4.4/0.3 |
| | | (−3.4) | (−5.6) | | (−0.8) | (−2.6) |

### EXAMPLE 5

This example illustrates the stability of anti-LILRB2 antibody variants under freeze-thaw stress.

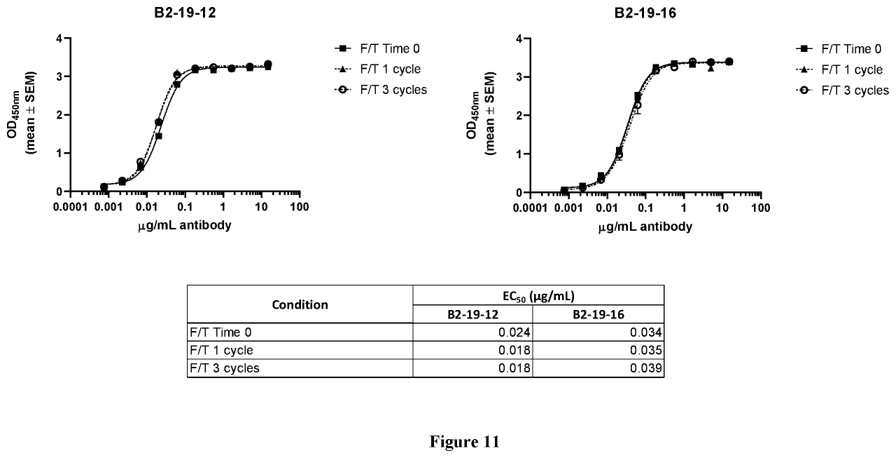

To assess the stability of B2-19-12 and B2-19-16 to freeze-thaw (F/T) stress, the antibodies were exposed to 1 (1 C) or 3 (3 C) cycles of freezing at −70° C. and thawing at room temperature (˜20° C.) at 20 mg/mL in formulation buffer FB (20 mM histidine-HCl, 7% sucrose, 0.02% w/v PS80, pH 5.5). Dynamic Light Scattering (DLS) was performed by WuXi Biologics which revealed no aggregative tendency as assessed by Z-ave (nm)/PDI (polydispersity index) (see Table 4). The activity of the antibodies before and after freeze-thaw cycles was assessed by ELISA. As shown in FIG. 11, no significant change in binding activity for either B2-19-12 or B2-19-16 was observed after freeze-thaw.

TABLE 4
B2-19 antibody variants do not show aggregative
tendency upon freeze-thaw.

DLS_Z-ave (nm)/PdI
F/T (−70° C./FT, ~20 mg/ml)

| Molecule | T0 | 1 C | 3 C |
|---|---|---|---|
| B2-19-12 | 24.68 (0.107) | 24.64 (0.111) | 24.74 (0.112) |
| B2-19-16 | 21.22 (0.096) | 21.27 (0.080) | 21.27 (0.096) |

### EXAMPLE 6

This example illustrates the pharmacokinetics of B2-19 antibody variants in human FcRn transgenic mice. A human FcRn transgenic mouse was used to assess pharmacokinetics (PK) of B2-19-12 and B2-19-16 since this model has shown the ability to predict antibody PK in humans (Avery et al mAbs 2016, 8, 1064). Specifically, 12 female mice Fcgrt^{mlDcr} homozygous, Tg(FCGRT)32Dcr homozygous (JAX stock #014565) aged 6-8 weeks were obtained from The Jackson Laboratory. Animals were dosed at 5 mg/kg in a single intravenous injection and 60 μL blood were collected following the schedule shown in Table 5. The concentrations of B2-19-12 and B2-19-16 in the serum were measured using a sandwich ELISA format. Briefly, the assay utilized recombinant LILRB2 ECD-6× His tagged protein coated on a 96-flat well microtiter plate as the capture reagent. The diluted samples and B2-19-12 or B2-19-16 standards were added to the coated plate. HRP-conjugated goat anti-human IgG was used as the detection reagent, resulting in an immune-complex with B2-19-12 or B2-19-16. Serum concentration-time plots for B2-19-12 and B2-19-16 were generated (FIG. 12). Pharmacokinetics parameters (Table 6) indicate similar exposure for B2-19-12 and B2-19-16 and that the terminal half-life and clearance are within typical ranges for human IgG in FcRn transgenic mice.

TABLE 5
Blood collection schedule for pharmacokinetics study in human FcRn transgenic
mice. “X” indicates when blood samples were harvested from each mouse cohort.

| | | Time point of blood collection post dose | | | | | | |
|---|---|---|---|---|---|---|---|---|
| Antibody | Cohort | 15 minutes | 6 hours | 24 hours | 72 hours | 120 hours | 168 hours | 336 hours |
| B2-19-12 | A (N = 3 mice) | X | | X | | X | | X |
| (1 mg/mL) | B (N = 3 mice) | | X | | X | | X | |
| B2-19-16 | C (N = 3 mice) | X | | X | | X | | X |
| (1 mg/mL) | D (N = 3 mice) | | X | | X | | X | |

TABLE 6
PK parameters for B2-19-12 and B2-19-16 in human FcRn transgenic mice

| | HL_Lambda_z | CL_obs | | AUClast | AUCINF_obs | AUC_%Extrap_obs | Cmax | Vss_obs |
|---|---|---|---|---|---|---|---|---|
| Antibody | day | mL/day/kg | mL/h/day | day*μg/mL | day*μg/mL | % | μg/mL | mL/kg |
| B2-19-12 | 9.41 | 12.46 | 0.52 | 270.35 | 401.21 | 32.62 | 57.15 | 159.02 |
| B2-19-16 | 12.19 | 9.65 | 0.40 | 290.24 | 518.37 | 44.01 | 62.10 | 164.76 |

### EXAMPLE 7

This example illustrates the characterization of the biological activity of B2-19 antibody variant B2-19-16 on multiple primary immune cell systems.

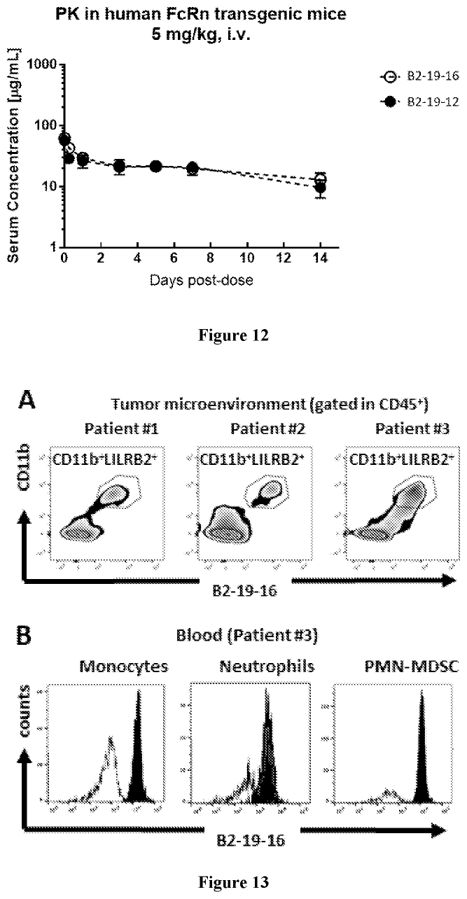

Tissue samples from solid tumors were dissociated into singles cells using mechanical methods and PBS-10 mM EDTA. In some cases, a peripheral blood sample was also obtained from same donor as tumor tissue sample. The resultant cells were stained with B2-19-16 and antibodies for markers of human myeloid cells at 4° C. and using standard methods and the stained samples were analyzed by flow cytometry. As shown in FIGS. 13A and 13B, B2-19-16 binds to all myeloid cells infiltrating solid tumor microenvironment, as well as peripheral blood myeloid cells from solid tumor patients. CD1 lb is used as pan-myeloid cell marker and CD45 is used as pan-tumor-infiltrating leukocytes marker.

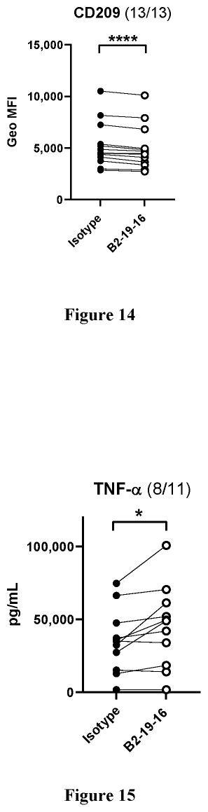

Classical monocytes were isolated from healthy donor PBMC and differentiated into immature dendritic cells with GM-CSF and IL-4 for 6 days. The immature monocyte-derived dendritic cells were then incubated with antibodies (100 nM) in the presence of 100 ng/mL LPS to induce dendritic cell maturation. After 2 days, the levels of TNF-α were measured in the media supernatant and the cells were analyzed by flow cytometry. As shown in FIG. 14, B2-19-16 antibody treatment led to a decrease in the expression levels of the tolerogenic marker CD209. In addition, as shown in FIG. 15, in comparison to isotype-treated condition, B2-19-16 further enhanced LPS-triggered TNF-α production. These results indicate that B2-19-16 potentiates the pro-inflammatory effect of LPS on immature monocyte-derived DC. This data suggests that LILRB2 blockade by B2-19-16 relieves the inhibitory signaling of LILRB2 on DC activating pathways, such as TLR.

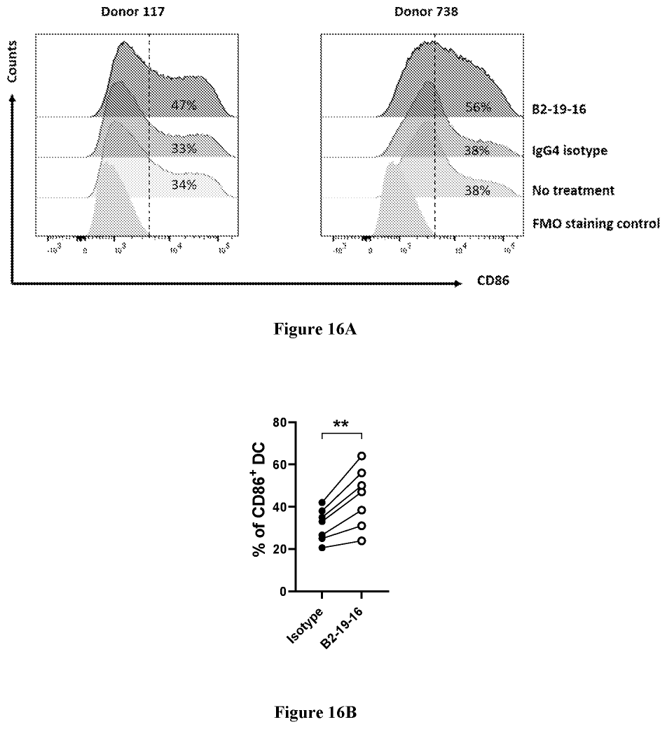

Classical monocytes were isolated from freshly prepared PBMC and treated with 15 μg/mL of B2-19-16 or isotype control, during a 6-day culture in 3 mL complete DC medium (StemXVivo Dendritic Cell Base Media, 50 μg/mL gentamicin, 50 ng/mL GM-CSF, 35 ng/mL IL-4) at a density of 1×10⁶ cells/mL, using 6-well plates. GM-CSF (50 ng/mL) and IL-4 (35 ng/mL) were added again on day 3. On day 6, the resulting DCs were analyzed by flow cytometry. As shown in FIGS. 16A and 16B, treatment of primary monocytes with B2-19-16 promoted their differentiation into pro-inflammatory (CD86⁺) DCs. This finding is key, as various tumor microenvironment-associated myeloid cell populations, including tolerogenic DCs, derive from circulating monocytes and it would be desirable to reprogram these cells into pro-inflammatory.

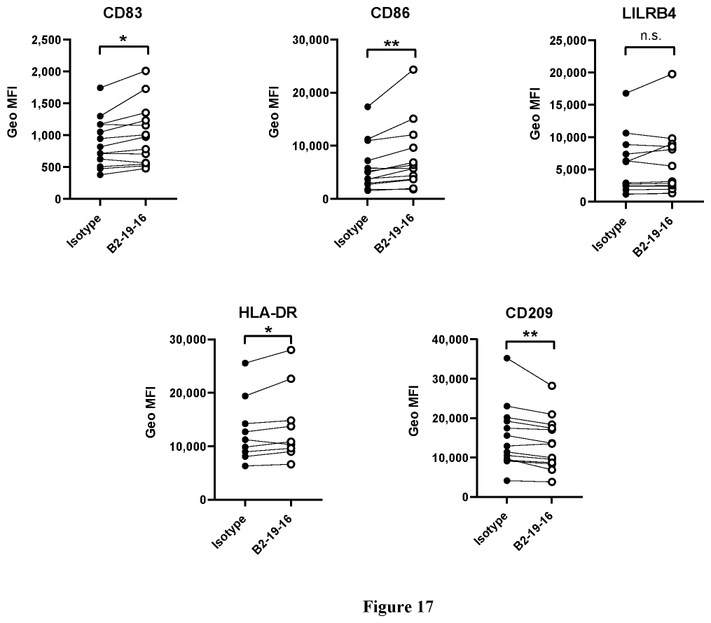

Classical monocytes were isolated from healthy donor PBMC and differentiated into immature dendritic cells with GM-CSF and IL-4 for 6 days. The immature monocyte-derived DCs were then incubated with antibodies (100 nM) in the absence of any other stimulus and their phenotype analyzed by flow cytometry after 2 days. As shown in FIG. 17, B2-19-16 enhances the expression levels of maturation (CD83) and activation markers (CD86, HLA-DR) in immature DCs while decreasing expression of the tolerogenic marker CD209. In contrast, the cell surface expression levels of LILRB4, another immune inhibitory receptor, remained unchanged. Therefore, B2-19-16 promotes the differentiation of immature DCs into DCs displaying enhanced ability to trigger adaptive immunity.

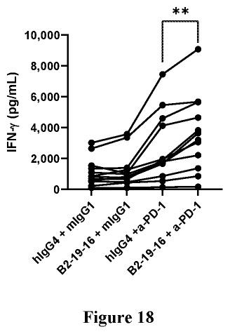

Macrophages were differentiated from classical monocytes isolated from PBMC of healthy donors with 100 ng/mL M-CSF for 6 days. On the sixth day, CD4⁺ T cells were isolated from PBMC of healthy, unrelated, donors and suspended in fresh media containing 100 ng/mL M-CSF and antibodies (100 nM each). This mixture was then added to the differentiated macrophages. At the end of 6 days, INF-γ levels in media supernatant were measured by ELISA. Anti-PD-1 antibody used is clone EH12.2H7. As shown in FIG. 18, B2-19-16 combined with anti-PD-1 blocking antibody enhances IFN-γ production in allogeneic CD4⁺ T cell-macrophage co-cultures, as compared to each antibody alone.

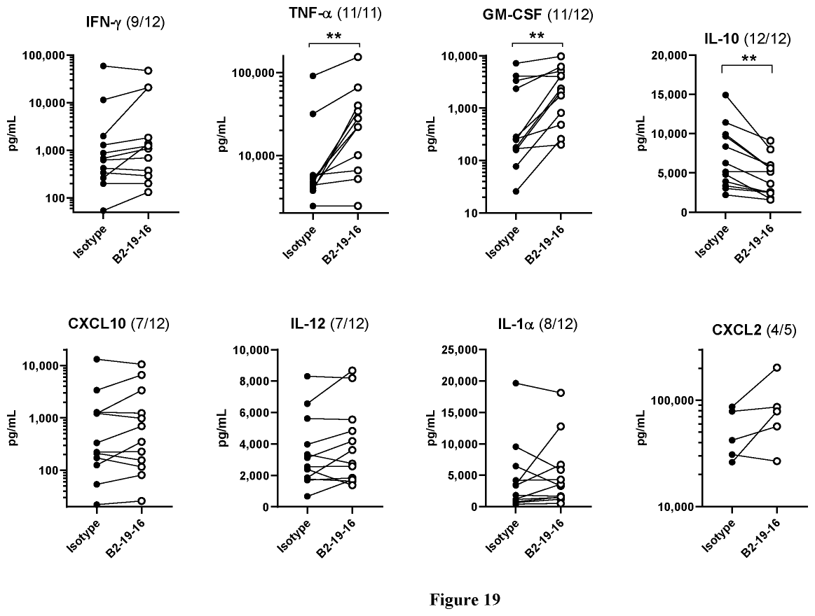

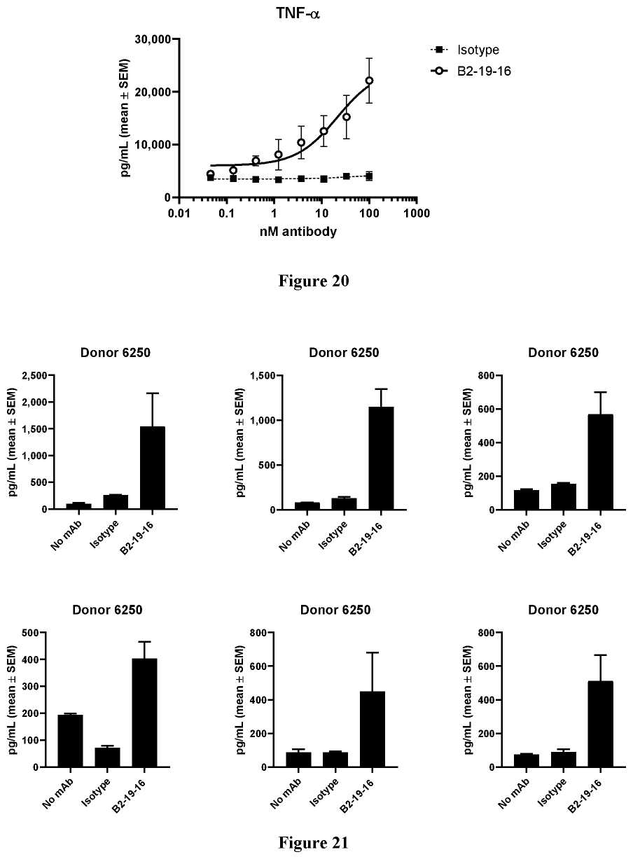

PBMC were isolated from healthy donors using Ficoll-Paque (GE Healthcare) and incubated in 96-well round bottom plates for 3 days with 50 ng/mL LPS in the presence of a 3-fold dilution series of anti-LILRB2 antibody B2-19-16 or isotype control. Cytokine concentration levels were measured in the culture media supernatant at the end of a 3-day incubation using a Human Cytokine Premixed Magnetic Luminex Performance Assay. As shown in FIG. 19, B2-19-16 antibody enhanced the concentration levels of various pro-inflammatory cytokines produced in response to LPS stimulus. Additionally, as shown for TNF-α in FIG. 20, B2-19-16 antibody enhances cytokine production by LPS-stimulated PBMC in a dose-dependent manner.

Classical monocytes were isolated from freshly prepared PBMC using the classical monocyte isolation kit. Classical monocytes were cultured in complete macrophage medium (X-VIVO 10, 4 mM of L-glutamine, 0.5 mg/mL penicillin/streptomycin and 100 ng/ml of M-CSF) at a density of 1×10⁶ cells/mL (100 μL/well) in flat-bottom 96-well plates. The cells were differentiated into macrophages for 7 days, with a media change on day 4. On day 7, media were replaced with complete macrophage medium containing 5 μg/mL of the STING agonist 2′3′ -cGAMP and 151.tg/mL of B2-19-16 or isotype control and the incubation continued for 2 days. The levels of TNF-α accumulated in the media during this 2-day incubation were measured using a human cytokine Luminex assay. As shown in FIG. 21, B2-19-16 potentiates the stimulatory effect of the cGAS-STING pathway in monocyte-derived macrophages from all tested donors, as indicated by the increased concentration levels of TNF-α.

The cancer cell lines SK-MEL-5 and A549 were seeded in 200 μL of culture medium (DMEM, 10% heat-inactivated FBS) at a density of 1×10⁴/mL in flat bottom, 96-well plates. The following day, myeloid cells were isolated from freshly prepared PBMC samples obtained from healthy donors using CD33 MicroBeads. The media of cancer cell cultures were replaced with 200 μl of co-culture medium (X-VIVO 10, 5% FBS, 50 ng/ml GM-CSF), containing 1×10⁵ myeloid cells and 15 μg/mL B2-19-16 or isotype control. Myeloid cells seeded in wells (1×10⁵/well) without cancer cells were used as a control for the effect of tumor conditioning. The cultures were maintained for 5 days. On day 5, cells were detached using 10 mM EDTA in PBS, and analyzed by flow cytometry. Myeloid cells were gated from live cells (7-AAD) based on forward scatter (FSC) and side scatter (SSC) signals and CD11b expression. The myeloid cell phenotype was evaluated by measuring changes in the expression levels of cell surface markers associated with either anti-inflammatory (CD163 and CD209) or pro-inflammatory activity (CD64). As shown in FIGS. 22A and 22B, “tumor conditioning” of myeloid cells led to up-regulated expression of the inhibitory receptor CD209 and of the scavenger receptor CD163. In contrast, the expression of the Fcγ receptor CD64 was decreased by tumor conditioning, indicating that the resulting macrophages display reduced antibody-mediated phagocytic ability. The presence of B2-19-16 in these cancer cell-myeloid cell co-cultures reverted these changes, suggesting that B2-19-16 treatment may preserve the pro-inflammatory and phagocytic potential of myeloid cells in cancer. Importantly, the activity of B2-19-16 is observed in the presence of cancer cell lines of distinct histological origin, suggesting that B2-19-16 may offer broad therapeutic benefit.

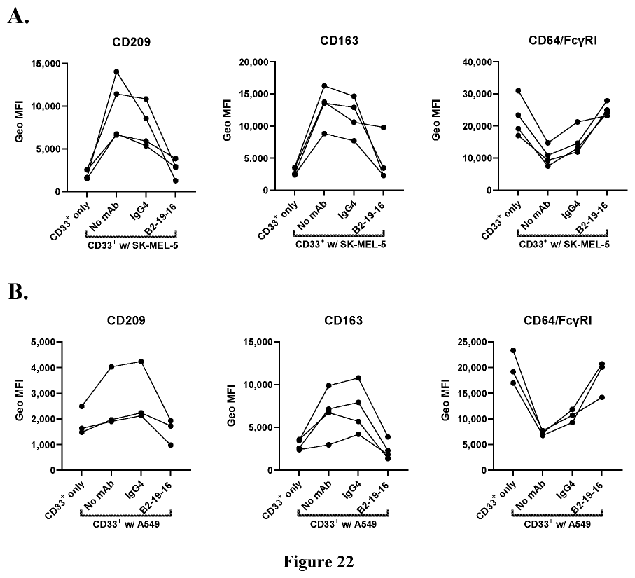

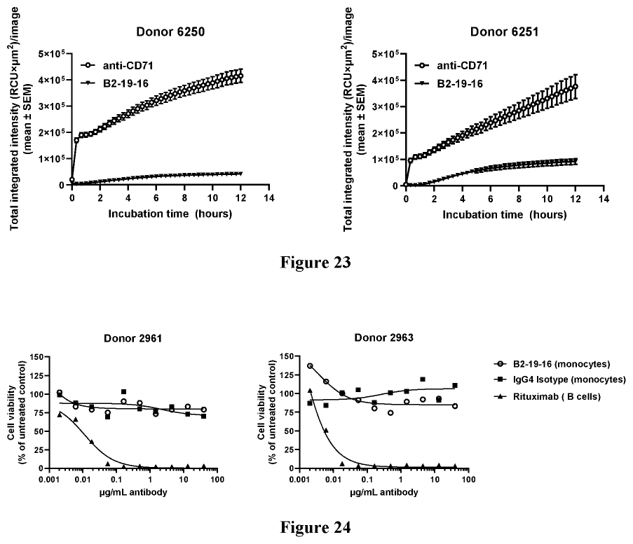

Classical monocytes were purified from freshly prepared PBMC using the classical monocyte isolation kit. Classical monocytes were cultured in complete macrophage medium (X-VIVO 10, 4 mM L-Glutamine, 0.5 mg/mL penicillin/streptomycin, 100 ng/ml M-CSF) at a density of 1×10⁶ cells/mL (100 μL/well) in flat-bottom 96-well plates. The cells were differentiated into macrophages for 7 days, with a media change on day 4. On day 7, B2-19-16 was labeled with the Fabfluor-pH Red Antibody Labeling Dye. The fluorescence emission levels of this dye increase at low pH. An anti-CD71 (transferrin receptor) antibody was also labeled in parallel and used as a positive control for internalization of receptor:antibody complexes. Following addition of antibody:FabFluor complexes (final concentration: 4 μg/ml) to cells, in triplicate wells, assay plates were placed in an Incucyte S3 live-cell analysis system (Essen Bioscience) and immediately scanned for phase and red fluorescence images at 10× magnification and then every 20 min for 12 hours. Images were analyzed using the Incucyte software for total integrated intensity to determine the levels of FabFluor labeled internalized antibody. As shown in FIGS. 23, whereas anti-CD71 antibody is efficiently internalized (increased fluorescence over time detected in the cells), B2-19-16 is not.

PBMC (1×10⁶ cells/well) were plated in a total volume of 200 μL X-VIVO 10 medium supplemented with 50 ng/ml IL-2 and a 3-fold dilution series (40-0.002m/mL) of B2-19-16, IgG4 (isotype control) or rituximab (positive control), using U-bottom 94-well plates. For each donor, PBMC plated as above, but in the absence of antibodies, were used as its respective untreated control. Cells were incubated for 20 hours at 37° C. Cells were washed with PBS and incubated with 400m/mL human IgG diluted in FACS buffer [PBS, 0.5% (wt/vol) BSA] (50 μL/well) at room temperature for 10 min. to block Fcγ receptors. Immediately after, an antibody cocktail (anti-CD14, clone M5E2 and anti-CD19, clone HIB19) diluted in FACS buffer was added (50 μL/well), mixed with the cell suspension by pipetting, and the cells were incubated on ice for 30 minutes. Cells were washed with FACS buffer and resuspended in 100 pL PBS containing 7-AAD diluted 1:20 (vol/vol). Cells were acquired in a BD FACSCelesta flow cytometer fitted with a high-throughput sample acquisition module and data from lx10 5 cells was recorded. Data was analyzed using FlowJo 10.5.3. Live cells were identified by exclusion of 7-AAD. Within the live PBMC population, monocytes were identified as CD14⁺ cells, whereas B cells were identified as CD19⁺ cells. For each cell type, cell viability at a given antibody concentration was calculated as percent of the untreated control value obtained for the same PMBC donor. Antibody concentration-cell viability curves were generated by plotting data in GraphPad Prism 9.1.0. As shown in FIG. 24, B2-19-16 does not trigger Fc-dependent depletion of monocytes. In contrast, depletion of B cells is observed in a parallel incubation of the same donor samples in the presence of rituximab.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. An anti-LILRB2 antibody or an antigen-binding fragment thereof, comprising a clone-paired heavy chain variable region and light chain variable region as set forth in FIG. 1.

2. The antibody or antigen-binding fragment thereof of claim 1, wherein
(a) the heavy chain variable region has the amino acid sequence of SEQ ID NO: 25 and the light chain variable region has the amino acid sequence of SEQ ID NO: 26; or
(b) the heavy chain variable region has the amino acid sequence of SEQ ID NO: 31 and the light chain variable region has the amino acid sequence of SEQ ID NO: 32.

3. The antibody or antigen-binding fragment thereof of claim 1, further comprising an immunoglobulin constant region, optionally a constant region of IgG, or optionally a constant region of human IgG, or optionally a constant region of IgG4, or optionally a constant region of hinge-stabilized IgG4.

4. The antibody or antigen-binding fragment thereof of claim 1, which is a humanized or fully human antibody.

5. The antibody or antigen-binding fragment thereof of claim 1, which is a camelized single domain antibody, a diabody, a scFv, a scFv dimer, a BsFv, a dsFv, a (dsFv)₂, a dsFv-dsFv′, an Fv fragment, a Fab, a Fab′, a F(ab′)₂, a bispecific antibody, a ds-diabody, a nanobody, a domain antibody, or a bivalent antibody.

6. The antibody or antigen-binding fragment of claim 1, which blocks the binding of LILRB2 to one or more ligand, wherein the ligand is selected from the group consisting of HLA-G, classical MHC-I, ANGPTLs, CD1c/d, CSPs and SEMA4A.

7. (canceled)

8. The antibody or antigen-binding fragment thereof of claim 1, which modulates the activation of LILRB2, wherein which suppresses the activation of LILRB2 or antagonizes LILRB2 signaling.

9-10. (canceled)

11. The antibody or antigen-binding fragment thereof of claim 1, which is multi-specific, wherein which binds specifically to a second antigen selected from PD-1, PD-L1, PD-L2, CTLA-4, LAG3, TIM-3, Fc receptors, FCRL(1-6), A2AR, CD160, 2B4, TGF-β, TGF-βR, VISTA, BTLA, TIGIT, LAIR1, LILRB1, LILRB3, LILRB4, LILRB5, IILRA(1-6), OX40, CD2, CD27, CD28, CD30, CD40, CD47, SIRPA, CLEC-1, clever-1/stabilin-1, ADGRE, TREM1, TREM2, CD122, ICAM-1, IDO, NKG2D/C, SLAMF7, M S4A4 A, SIGLEC(7-15), NKp80, NKG2A, CD160, CD161, CD300, CD163, B7-H3, B7-H4, LFA-1, ICOS, 4-1BB, GITR, BAFFR, HVEM, CD7, LIGHT, TNFR2, TLR(1-9), 1L-2, 1L-7, 1L-15, 1L-21, CD16 and CD83.

12. (canceled)

13. The antibody or antigen-binding fragment thereof of claim 1, which is linked to one or more conjugate moieties, wherein the conjugate moiety comprises an immune modulatory agent, an anti-tumor drug, a clearance-modifying agent, a toxin, a detectable label, a DNA, an RNA, a cytokine, or purification moiety.

14. (canceled)

15. A pharmaceutical composition comprising the antibody or antigen-binding fragment thereof of claim 1, and a pharmaceutically acceptable carrier.

16. An isolated polynucleotide encoding the antibody or antigen-binding fragment thereof of claim 1.

17. A vector comprising the isolated polynucleotide of claim 16.

18. A host cell comprising the vector of claim 17.

19. A method of expressing an antibody or antigen-binding fragment thereof, comprising culturing the host cell of claim 18 under the condition at which the antibody or antigen-binding fragment thereof is expressed.

20. A chimeric antigen receptor (CAR) protein comprising an antigen-binding fragment according to claim 1.

21. An isolated nucleic acid that encodes a CAR protein of claim 20.

22. A vector comprising the isolated nucleic acid of claim 21.

23. An engineered cell comprising the isolated nucleic acid of claim 21, wherein the cell is a T cell, NK cell, or macrophage.

24. (canceled)

25. A method of treating or ameliorating the effect of a cancer in a subject, comprising administering to the subject a therapeutically effective amount of the antibody or antigen-binding fragment thereof of claim 1.

26-38. (canceled)

39. A method of detecting a cancer cell or cancer stem cell in a sample or subject, wherein the sample is a body fluid or biopsy, optionally wherein the sample is blood, bone marrow, sputum, tears, saliva, mucous, serum, ascites, urine or feces, comprising:
(a) contacting a subject or a sample from the subject with the antibody or an antigen-binding fragment thereof according to claim 1; and
(b) detecting binding of said antibody to a cancer cell or cancer stem cell in said subject or sample; optionally
(c) performing steps (a) and (b) a second time and determining a change in detection levels as compared to the first time.

40-46. (canceled)

47. A method for enhancing T cell activation or enhancing dendritic cell maturation and activation, modulating anti-inflammatory macrophage and tolerogenic DC phenotype; polarizing myeloid cells from solid tumor cancer patients towards a pro-inflammatory phenotype, or alleviating suppressive effect of patient-derived monocytic MDSC (M-MDSC) on autologous T cell proliferation and cytokine release in a subject, the method comprising administering to the subject the antibody or an antigen-binding fragment thereof according to claim 1.

48-50. (canceled)
